(19) United States
(12) Patent Application Publication
(10) Pub. No.: US 2003/0087865 A1
(43) Pub. Date: May 8, 2003

(54) Leukemogenic transcription factors

(75) Inventors: Todd R. Golub, Newton, MA (US); Koichi Sasaki, Osaka (JP); Robert V. Martinez, Roslindale, MA (US); Eric S. Lander, Cambridge, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA

(21) Appl. No.: 10/196,332

(22) Filed: Jul. 15, 2002

Related U.S. Application Data
(60) Provisional application No. 60/305,554, filed on Jul. 13, 2001.

(51) Int. Cl. A61K 48/00; C12Q 1/68; G01N 33/574; C07H 21/04; C12N 9/22

(52) U.S. Cl. 514/44; 435/6; 536/23.2; 435/7.23; 435/199

(57) ABSTRACT

A previously unrecognized level of transcriptional control by the leukemogenic transcription factors TEL, and AML1/ETO, of the interferon gamma signaling pathway has been discovered. Gene expression analysis has identified downstream targets of these leukemogenic transcription factors. The associated expression and regulation of these genes in leukemia, and methods of use thereof, are described herein.

### GOVERNMENT SUPPORT

The invention was supported in part by the National Institutes of Health, grant number P01 CA72009-01A1. The Government has certain rights in the invention.

### RELATED APPLICATION

This application claims the benefit of U.S. Provisional Application No. 60/305,554, filed on Jul. 13, 2001, the entire teachings of which are incorporated herein by reference.

### BACKGROUND OF THE INVENTION

Hematopoiesis is the coordinated generation of mature cells of the blood from rare stem cells in the bone marrow of adults. It is controlled by key transcription factors, which regulate specific genes involved in lineage determination and differentiation of pluripotent cells. Many of these transcription factors are recognized to be mutated and frequently rearranged by chromosomal translocation in leukemia. However, it is not known how these mutated and rearranged transcription factors function. Therefore, there is a need to identify the transcriptional targets of these transcription factors, and thus elucidate the role of these transcription factors in the generation of leukemia.

### SUMMARY OF THE INVENTION

Work described herein details the identification of downstream targets of the leukemogenic transcription factors TEL and the fusion protein AML1/ETO. This work has discovered that both proteins dysregulate the interferon gamma transcriptional response, by repressing the expression of the interferon regulatory factor ICSBP (interferon consensus sequence binding protein). Gene expression analysis of hematopoietic cells expressing AML1/ETO described herein has demonstrated that these cells fail to induce MHC class 2 molecules or the co-stimulatory molecule B7-1 in response to interferon gamma stimulation. Interferon gamma has a cytostatic effect on myeloid cells that is blocked by cells over-expressing either AML1/ETO or TEL, with resulting repression of ICSBP. Reintroduction of ICSBP into TEL-overexpressing cells restores the cytostatic effect of interferon gamma, indicating that ICSBP is the critical target of TEL regulation. Transcriptional profiling of TEL-expressing cells described herein has identified a number of genes that are regulated by TEL, including calcyclin, IL-6, an EST similar to yeast YER036C and Id1. Expression of both calcyclin and IL-6 were elevated in TEL expressing cells, whereas expression of the EST similar to yeast YER036C and Id1 were repressed. Expression of AML-1/ETO is associated with decreased expression of ICSBP, MHC class 2 and B7-1.

The invention relates to a method for diagnosing a disorder associated with expression of a leukemogenic transcription factor, comprising determining the expression of one or more target genes of the leukemogenic transcription factor.

In one embodiment, the leukemogenic transcription factor is TEL, a functional fragment of TEL, TEL fusion protein, or AML1/ETO.

In another embodiment, the expression of the leukemogenic transcription factor results in the dysregulation of the interferon gamma transcriptional response.

In a further embodiment, the expression of the one or more target genes is determined by assessing mRNA levels of said genes.

Alternatively, the expression of the one or more target genes is determined by assessing protein levels of said genes.

In another embodiment, the expression of a leukemogenic transcription factor is altered, including loss of TEL expression, overexpression of TEL, expression of TEL fragments, expression of TEL fusion genes associated with chromosomal translocations, or expression of AML1/ETO.

In an additional embodiment, the disorder associated with expression of a leukemogenic transcription factor is a lymphoproliferative disorder, lymphoid leukemia, myeloid leukemia, acute leukemia or chronic leukemia.

In a further embodiment, the method for diagnosing a disorder associated with the expression of TEL, a functional fragment of TEL, or a TEL fusion protein, comprises determining the expression levels of ICSBP, Id1, IL-6, calcyclin and/or an EST similar to yeast YER036C.

In another embodiment, the expression of TEL, a fragment of TEL, or TEL fusion proteins, is associated with repressed promoter activity of ICSBP. Alternatively, or additionally, the expression of TEL, a fragment of TEL, or TEL fusion proteins, is associated with deacetylation of Histone H3. Furthermore, the expression of TEL, a fragment of TEL, or of TEL fusion proteins, can be associated with repressed expression of one or more genes selected from the group consisting of: ICSBP, Id1, and an EST similar to yeast YER036C. In another embodiment, the expression of TEL, a fragment of TEL, or TEL fusion proteins, is associated with elevated expression of IL-6 and/or calcylcin.

In another embodiment, the method for diagnosing a disorder associated with the expression of AML1/ETO, comprises determining the expression levels of ICSBP, MHC class 2, and/or B7-1. In a further embodiment, the expression of AML/ETO is associated with repressed expression of ICSBP, MHC class 2, and/or B7-1.

The invention also relates to a method of screening for an agent that is an agonist, mimic, or antagonist of TEL, comprising the steps of culturing cells that express TEL in a suitable medium, introducing the test agent to TEL-expressing cells and assaying TEL-expressing cells for altered expression of ICSBP, Id1, IL-6, calcyclin or EST similar to yeast YER036C. Similarly, control cells, which do not express TEL, are cultured in a suitable medium, and are introduced to the same test agent, and similarly assaying the control cells for expression of ICSBP, Id1, IL-6, calcyclin or EST similar to yeast YER036C, wherein repressed expression of ICSBP, Id1, EST similar to yeast YER036C, or increased expression of IL-6 or calcyclin in cells that express TEL and treated with the agent, relative to control cells treated with the agent, indicates that the agent is an agonist or mimic of TEL, whereas increased expression of ICSBP, Id1, EST similar to yeast YER036C, or repressed expression of IL-6 or calcyclin in cells that express TEL and treated with the agent, relative to control cells treated with the agent, indicates that the agent is an antagonist of TEL.

In another embodiment, a method of screening for an agent that is an agonist, mimic, or antagonist of TEL, comprises the steps of culturing cells that express TEL in a suitable medium, introducing an agent to be tested to TEL-expressing cells, assaying TEL-expressing cells for altered expression of ICSBP, Id1, IL-6, calcyclin or EST similar to yeast YER036C, and assaying TEL-expressing cells for basal expression levels of ICSBP, Id1, IL-6, calcyclin or EST similar to yeast YER036C, wherein repressed expression of ICSBP, Id1, EST similar to yeast YER036C, or increased expression of IL-6 or calcyclin in cells that express TEL and treated with the agent, relative to basal levels, indicates that the agent is an agonist or mimic of TEL, whereas increased expression of ICSBP, Id1, EST similar to yeast YER036C, or repressed expression of IL-6 or calcyclin in cells that express TEL and treated with the agent, relative to basal levels, indicates that the agent is an antagonist of TEL.

The invention also relates to a method for determining the effectiveness of an agent that modulates TEL activity for treatment of a disorder characterized by altered TEL expression, comprising determining the differentiation of test cells that have altered TEL expression, cultured in a suitable culture medium in the presence and absence of the agent to be tested, wherein the test cells are obtained from the subject, and increased differentiation of test cells in the presence of the agent, relative to test cells in the absence of the agent, is predictive of the efficacy of the agent for the treatment of the disorder.

In one embodiment, the method for determining the effectiveness of an agent that modulates TEL activity uses test cells that are selected from the group consisting of hematopoietic cells, bone marrow-derived cells, splenocytes, and circulating lymphocytes.

The invention also relates to a method for determining the effectiveness of an agent that modulates TEL activity for treatment of a disorder characterized by altered TEL expression, comprising determining the proliferation of test cells that have altered TEL expression, cultured in a suitable culture medium in the presence and absence of the agent to be tested, wherein the test cells are obtained from the subject, and decreased proliferation of test cells in the presence of the agent, relative to test cells in the absence of the agent, is predictive of the efficacy of the agent for the treatment of the disorder.

In one embodiment, the method for determining the effectiveness of an agent that modulates TEL activity uses test cells that are selected from the group consisting of hematopoietic cells, bone marrow-derived cells, splenocytes, and circulating lymphocytes.

Also provided in the invention is a method of screening for an agent that is an agonist, mimic, or antagonist of AML1/ETO, comprising the steps of culturing cells that express AML1/ETO in a suitable medium, introducing an agent to be tested to AML1/ETO-expressing cells, assaying AML1/ETO-expressing cells for altered expression of ICSBP, MHC class 2, or B7-1, and culturing control cells, which do not express AML1/ETO, in a suitable medium, introducing same said agent to be tested, and assaying said control cells for expression of ICSBP, MHC class 2, or B7-1, wherein repressed expression of ICSBP, MHC class 2, or B7-1 in cells that express AML1/ETO and treated with the agent, relative to control cells treated with the agent, indicates that the agent is an agonist or mimic of AML1/ETO, and whereas increased expression of ICSBP, MHC class 2, or B7-1 in cells that express AML1/ETO and treated with the agent, relative to control cells treated with the agent, indicates that the agent is an antagonist of AML1/ETO.

Additionally, the invention provides a method of screening for an agent that is an agonist, mimic, or antagonist of AML1/ETO, comprising the steps of culturing cells that express AML1/ETO in a suitable medium, introducing an agent to be tested to AML1/ETO-expressing, assaying AML1/ETO-expressing cells for altered expression of ICSBP, MHC class 2, or B7-1, and assaying AML1/ETO-expressing cells for basal expression levels of ICSBP, MHC class 2, or B7-1, wherein repressed expression of ICSBP, MHC class 2, or B7-1 in cells that express AML1/ETO and treated with the agent, relative to basal expression levels, indicates that the agent is an agonist or mimic of AML1/ETO, and whereas increased expression of ICSBP, MHC class 2, or B7-1 in cells that express AML1/ETO and treated with the agent, relative to basal expression levels, indicates that the agent is an antagonist of AML1/ETO.

Also provided in the invention is a method for determining the effectiveness of an agent that modulates AML1/ETO activity for treatment of a disorder characterized by altered AML1/ETO expression, comprising determining the differentiation of test cells that have altered AML1/ETO expression, cultured in a suitable culture medium in the presence and absence of the agent to be tested, wherein the test cells are obtained from the subject, and increased differentiation of test cells in the presence of the agent, relative to test cells in the absence of the agent, is predictive of the efficacy of the agent for the treatment of the disorder.

In one embodiment, the method for determining the effectiveness of an agent that modulates AML1/ETO activity uses test cells that are selected from the group consisting of haematopoietic cells, bone marrow-derived cells, splenocytes, and circulating lymphocytes.

The invention further relates to a method for determining the effectiveness of an agent that modulates AML1/ETO activity for treatment of a disorder characterized by altered AML1/ETO expression, comprising determining the proliferation of test cells that have altered AML1/ETO expression, cultured in a suitable culture medium in the presence and absence of the agent to be tested, wherein the test cells are obtained from the subject, and decreased proliferation of test cells in the presence of the agent, relative to test cells in the absence of the agent, is predictive of the efficacy of the agent for the treatment of the disorder.

In one embodiment, the method for determining the effectiveness of an agent that modulates AML1/ETO activity uses test cells that are selected from the group consisting of haematopoietic cells, bone marrow-derived cells, splenocytes, and circulating lymphocytes.

The invention also relates to a method of treatment of an individual having a disorder characterized by one or more parameters including (a) elevated or repressed TEL gene expression, (b) expression of TEL protein, or fragment thereof, fused to another protein as a consequence of chromosomal translocation, (c) elevated or repressed ICSBP gene expression, (d) elevated or repressed Id1 gene expression, (e) elevated or repressed calcyclin gene expression, (f) elevated or repressed EST similar to yeast YER036C gene expression, and (g) elevated or repressed IL-6 gene expression, wherein the method comprises administering to the individual an effective amount of an agent that modulates TEL activity.

In one embodiment, the disorder is lymphoid, myeloid, acute or chronic leukemia. Furthermore, the individual can be treated by administering the agent orally, intravenously, intramuscularly, subcutaneously, topically, rectally, or by inhalation.

Also provided in the invention is a method of treatment of an individual having a disorder characterized by one or more parameters including (a) expression of AML1/ETO, (b) elevated or repressed ICSBP gene expression, (c) elevated or repressed MHC class 2 gene expression, and (d) elevated or repressed B7-1 gene expression, such that the method comprising administering to the individual an effective amount of an agent that modulates AML1/ETO activity.

In one embodiment, the disorder is lymphoid, myeloid, acute or chronic leukemia. Furthermore, the individual can be treated by administering the agent orally, intravenously, intramuscularly, subcutaneously, topically, rectally, or by inhalation.

The invention also relates to a method for diagnosing a disorder associated with altered leukemogenic transcription factor expression, comprising determining the expression level of one or more target genes of the leukemogenic transcription factor, wherein the disorder is lymphoid leukemia, myeloid leukemia, acute leukemia, or chronic leukemia.

In a further embodiment, the method for diagnosing a disorder associated with altered leukemogenic transcription factor expression, is also associated with dysregulation of the interferon gamma transcriptional response.

In one embodiment, dysregulation of the interferon gamma transcriptional response is associated with expression of TEL, a functional fragment of TEL, TEL fusion proteins, or AML1/ETO.

In a further embodiment, the dysregulation of the interferon gamma transcriptional response is associated with lymphoid leukemia, myeloid leukemia, acute leukemia or chronic leukemia.

Also provided in the invention is a kit for diagnosis of a disorder associated with altered leukemogenic transcription factor expression which comprises one or more reagents to detect the expression of ICSBP, Id1, EST similar to yeast YER036C, IL-6, calcyclin, and combinations thereof. Additionally, or alternatively, a kit for diagnosis of a disorder associated with altered leukemogenic transcription factor expression can comprise one or more reagents to detect the expression of ICSBP, MHC class 2, B7-1, and combinations thereof.

The invention also relates to a method for inducing gene expression of IL-6 and/or calcyclin in a cell, by inducing the expression of TEL.

Furthermore, the invention relates to a method for inhibiting gene expression of ICSBP, Id1, and/or an EST similar to yeast YER036C in a cell, by inducing the expression of TEL in said cell.

Also provided in the invention is a method for inhibiting gene expression in a cell of IL-6 and/or calcyclin, by inhibiting the expression of TEL.

Furthermore, the invention provides a method to increase expression of interferon gamma-induced genes in a cell by administering to the cell an agent selected from the group consisting of ICSBP, an ICSBP mimic, an ICSBP agonist, and combinations thereof.

Additionally provided is a method to induce interferon gamma-induced cytostasis in a cell by administering to the cell an agent selected from the group consisting of ICSBP, an ICSBP mimic, an ICSBP agonist, and combinations thereof.

The invention also relates to a method of treatment of an individual having a disorder characterized by one or more parameters including, (a) elevated or repressed TEL gene expression, (b) expression of TEL protein, or fragment thereof, fused to another protein as a consequence of chromosomal translocation, (c) elevated or repressed ICSBP gene expression, (d) elevated or repressed Id1 gene expression, (e) elevated or repressed calcyclin gene expression, (f) elevated or repressed EST similar to yeast YER036C gene expression, (g) expression of AML1/ETO, (h) elevated or repressed MHC class 2 gene expression, (i) elevated or repressed B7-1 gene expression, and (j) elevated or repressed IL-6 gene expression, wherein said method comprising administering to the individual an effective amount of an agent that modulates ICSBP activity.

In one embodiment, administration of an agent that modulates ICSBP activity is to an individual with a disorder that is lymphoid, myeloid, acute or chronic leukemia. Administration of the agent can be orally, intravenously, intramuscularly, subcutaneously, topically, rectally, or by inhalation.

Furthermore, the invention relates to a method of treatment of an individual having a disorder characterized by repressed expression of genes normally induced by interferon gamma stimulation, by administering a therapeutically-effective amount of an agent selected from the group consisting of: ICSBP, an ICSBP mimic, an ICSBP agonist, and combinations thereof.

In one embodiment, the disorder characterized by repressed expression of genes normally induced by interferon gamma stimulation is associated with the expression of TEL, a functional fragment of TEL or TEL fusion protein. Alternatively or additionally, the disorder is associated with the expression of AML1/ETO.

In still another aspect, the invention features a method of identifying a compound that modulates the biological activity of TEL. The method comprises the steps of a) contacting TEL with a candidate compound under conditions suitable for activity of TEL; and b) assessing the biological activity level of TEL. A candidate compound that increases or decreases the biological activity level of TEL relative to a control is a compound that modulates the biological activity of TEL. In one embodiment, the method is carried out in a cell or animal. In another embodiment, the method is carried out in a cell free system. In still another embodiment the biological activity of TEL is the dysregulation of IFN gamma signaling.

In another aspect, the invention features a method of identifying a compound that modulates the biological activity of AML1/ETO. The method comprises the steps of a) contacting AML1/ETO with a candidate compound under conditions suitable for biological activity of AML1/ETO; and b) assessing the biological activity level of AML1/ETO. A candidate compound that increases or decreases the biological activity level of AML1/ETO relative to a control is a compound that modulates the biological activity of AML1/ETO. In one embodiment, the method is carried out in a cell or animal. In another embodiment, the method is carried out in a cell free system. In still another embodiment the biological activity of AML1/ETO is the dysregulation of IFN gamma signaling.

In another aspect, the invention features a method of identifying a compound that decreases expression of TEL. The method comprises the steps of a) providing a nucleic acid molecule comprising a promoter region of TEL, or part of such a promoter region, operably linked to a reporter gene; b) contacting the nucleic acid molecule with a candidate compound under conditions suitable for TEL promoter activity; and c) assessing the level of expression of the reporter gene. A candidate compound that decreases expression of the reporter gene relative to a control is a compound that decreases expression of TEL. In one embodiment, the method is carried out in a cell.

In another aspect, the invention features a method of identifying a compound that decreases expression of AML1/ETO. The method comprises the steps of a) providing a nucleic acid molecule comprising a promoter region of AML1/ETO, or part of such a promoter region, operably linked to a reporter gene; b) contacting the nucleic acid molecule with a candidate compound under conditions suitable for AML1/ETO promoter activity; and c) assessing the level of expression of the reporter gene. A candidate compound that decreases expression of the reporter gene relative to a control is a compound that decreases expression of AML1/ETO. In one embodiment, the method is carried out in a cell.

In another aspect, the invention features a method of identifying a compound that increases expression of ICSBP. The method comprises the steps of a) providing a nucleic acid molecule comprising a promoter region of ICSBP, or part of such a promoter region, operably linked to a reporter gene; b) contacting the nucleic acid molecule with a candidate compound under conditions suitable for ICSBP promoter activity; and c) assessing the level of expression of the reporter gene. A candidate compound that increases expression of the reporter gene relative to a control is a compound that increases expression of ICSBP. In one embodiment, the method is carried out in a cell.

In still another aspect, the invention features a method of identifying a polypeptide that interacts with TEL. The method comprises the steps of a) providing a first nucleic acid vector comprising a nucleic acid molecule encoding a DNA binding domain and a polypeptide encoded by TEL; b) providing a second nucleic acid vector comprising a nucleic acid encoding a transcription activation domain and a nucleic acid encoding a test polypeptide; c) contacting the first nucleic acid vector with the second nucleic acid vector in a yeast two-hybrid system; and d) assessing transcriptional activation in the yeast two-hybrid system. An increase in transcriptional activation relative to a control indicates that the test polypeptide is a polypeptide that interacts with TEL.

In still another aspect, the invention features a method of identifying a polypeptide that interacts with AML1/ETO. The method comprises the steps of a) providing a first nucleic acid vector comprising a nucleic acid molecule encoding a DNA binding domain and a polypeptide encoded by AML1/ETO; b) providing a second nucleic acid vector comprising a nucleic acid encoding a transcription activation domain and a nucleic acid encoding a test polypeptide; c) contacting the first nucleic acid vector with the second nucleic acid vector in a yeast two-hybrid system; and d) assessing transcriptional activation in the yeast two-hybrid system. An increase in transcriptional activation relative to a control indicates that the test polypeptide is a polypeptide that interacts with AML1/ETO.

In still another aspect, the invention features a method of identifying a polypeptide that interacts with ICSBP. The method comprises the steps of a) providing a first nucleic acid vector comprising a nucleic acid molecule encoding a DNA binding domain and a polypeptide encoded by ICSBP; b) providing a second nucleic acid vector comprising a nucleic acid encoding a transcription activation domain and a nucleic acid encoding a test polypeptide; c) contacting the first nucleic acid vector with the second nucleic acid vector in a yeast two-hybrid system; and d) assessing transcriptional activation in the yeast two-hybrid system. An increase in transcriptional activation relative to a control indicates that the test polypeptide is a polypeptide that interacts with ICSBP.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the results of a western blot using anti-TEL antibody on two stable 32D lines (#1, #12) that over-express human TEL. The TEL protein appears as a doublet due to internal initiation of translation. On the same blot, Western hybridization with an anti-tubulin antibody was performed.

FIG. 2 shows the results of a Northern blot analysis of four target genes of TEL identified by oligonucleotide microarray. RNA was isolated independently to those hybridized on the microarray and then probed for calcyclin, IL-6, YER036C and Id1 expression. The blots reveal that both calcyclin and IL-6 are upregulated, whereas YER036C and Id1 are repressed by TEL.

FIG. 3A is a western analysis and shows the expression of Id1 protein decreases with TEL over-expression. The same blot probed with an anti-tubulin antibody reveals equal protein loading and integrity in each lane.

FIG. 3B is a western analysis and shows that constitutive expression of TEL in BaF3 cells is associated with reduced levels of Id1 protein. Stable TEL expressing BaF3 clones were generated and Western blot analysis reveals the relative level of TEL in three independent clones relative to the parental line as well as significantly reduced Id1 protein. The same blot probed for tubulin expression reveals relatively similar protein levels.

FIG. 4 shows the results demonstrating that TEL represses the transcriptional activity of the Id1 210 bp enhancer. Luciferase reporter plasmids were either wild-type 210-bp enhancer containing tandem repeats of a candidate EBS plasmid or plasmids containing mutations in the EBS, or a plasmid containing an irrelevant mutation with the EBS sites intact. The effector plasmids used were either the pcDNA3 vector containing either wild-type TEL cDNA ETS-2 or a TEL construct lacking the DNA binding domain (TELADBD). A total of 20 μg of DNA was electroporated into BaF3 cells and cells were harvested 48 hr post-electroporation and assessed for luciferase activity. The experiment was performed six times in triplicate, with similar results, and the average luciferase activity of one such experiment is shown with the error bars indicating standard deviations.

FIG. 5A is an EMSA analysis and shows that TEL potentiates the formation of the Ets-specific complex (*) within the Id1 210 bp enhancer. EMSA of nuclear extracts from both 32D parental and with enforced TEL mixed with wild type (Wt) or mutated (M) Id1 oligonucleotides reveal that the complex is prominently formed with exogenous TEL. Additionally, in competitive EMSA with 100-fold excess unlabelled wild type oligonucleotides, complex is diminished. Unlabelled mutated oligonucleotide has no effect on the labeled complex formation.

FIG. 5B is a western analysis and demonstrate that TEL binds in vivo to the Id 1 promoter. Chromatin immunoprecipitation was performed on parental 32D and TEL over-expressing 32D cells using a rabbit polyclonal anti-TEL antibody and controls (non-immune serum and beads alone). The immunoprecipitated DNA was then amplified using semi-quantitative PCR with primers spanning 200 bp of the mouse Id1 enhancer. Total genomic DNA was used as an input control. The specificity of the chromatin immunoprecipitation was confirmed by PCR amplification for tyrosinase, a gene not regulated by TEL.

FIG. 6 shows graphs of the results demonstrating that TEL represses ICSBP expression.

FIG. 7 shows the results that TEL represses interferon gamma-induced ICSBP induction. Parental 32D cells and TEL-expressing 32D cells were treated with interferon gamma and the expression of ICSBP determined. 32D cells strongly induce ICSBP expression, whereas 32D/TEL cells do not induce ICSBP expression. Over-expression of TEL was confirmed in 32D/TEL cells.

FIG. 8 is a model of interferon gamma signaling and TEL.

FIG. 9 demonstrates the results that STAT-1 phosphorylation is unaffected by TEL. Parental and TEL-overexpressing 32D cell lines #1 and #12 were tested in the presence and absence of interferon gamma for STAT-1 phosphorylation. No difference was detected in the phosphorylation status of STAT-1, whether TEL was expressed or not. Tubulin was used as a loading control.

FIG. 10 is the 5′ flanking region of the mouse ICSBP gene (SEQ ID NO: 1). The sequence of the 5′ flanking region of the mouse ICSBP gene contains two putative EBS (Ets-binding sequences), consensus STAT-1 binding site, CAAT signal and TATA signal sequences.

FIG. 11 is a graph charting the results that TEL represses the activity of the 5′ ICSBP flanking region. Using a Luciferase assay, cells repress the activity of the 5′ ICSBP flanking region when TEL is present, as compared to control pcDNA3. This repression by TEL is dependent on the DNA-binding domain (DBD) as transfection with a TEL construct without the DBD fails to repress activity of the 5′ ICSBP flanking region. TEL/AML1 also represses the activity of the 5′ ICSBP flanking region when compared to control pcDNA3.

FIG. 12 show the finding that TEL repression of ICSBP is due to specific deacetylation of Histone H3. Parental 32D cells or 32D/TEL cells in the presence or absence of interferon-gamma were subjected to cross-linking, homogenization, and the nuclei sonicated immunoprecipitations were performed with antibodies to TEL, Ets2, STAT-1, acetylated Histone H3, acetylated Histone H4, a control antibody, or without antibody. Immunoprecipitates were washed, the DNA eluted, and subjected to semi-quantitative polymerase chain reaction (PCR) for a region of the ICSBP EBS-containing sequence. Acetylated Histone H3 was associated with the EBS-containing region of ICSBP in parental 32D cells, but not in TEL expressing cells. TEL expression also inhibits the interferon gamma-induced STAT-1 association with the EBS-containing region of ICSBP.

FIG. 13 is a graph demonstrating the results that TEL suppresses the cytostatic effect of interferon gamma in 32D mycloid cells. After four days in culture, TEL-expressing cells can be seen to suppress the cytostatic effect of interferon gamma, as compared to parental 32D cells.

FIG. 14 is a schematic diagram of murine genome U74A Affymetrix gene chips to analyze the global effect of TEL on interferon gamma signaling

FIG. 15 is a graph showing the results that TEL represses interferon gamma-dependent ICSBP induction. In 32D cells, ICSBP is rapidly induced upon interferon gamma-dependent treatment. TEL suppresses the interferon gamma-dependent ICSBP induction.

FIG. 16 is a chart illustrating the finding that TEL significantly affects interferon gamma-induced genes. The expression profile of several genes were analyzed in 32D and 32D/TEL cells and compared. Two hundred and twenty four genes normally induced by interferon gamma treatment were suppressed in TEL-expressing cells, including ICSBP.

FIG. 17 is a graph showing the results that expression of ICSBP in 32D/TEL cells restores interferon gamma-induced cytostasis.

FIG. 18 is a chart showing that ICSBP expression in 32D/TEL cells rescues 100% of TEL-repressed interferon gamma-induced genes. Three hundred and sixty-eight genes normally repressed in TEL-expressing cells were induced in response to interferon gamma in 32D/TEL/ICSBP cells, including ICSBP.

The foregoing and other objects, features and advantages of the invention will be apparent from the following more particular description of preferred embodiments of the invention.

### DETAILED DESCRIPTION OF THE INVENTION

Hematopoiesis is the coordinated generation of mature cells of the blood from rare stem cells in the bone marrow of adults. It is controlled by key transcription factors, which regulate specific genes involved in lineage determination and differentiation of pluripotent cells. Master regulators of hematopoiesis include SCL, GATA-1, GATA-2 and LMO-2. Other transcription factors, which are required for the development of defined lineages include c-MYB, PU.1, PAX-5, Ikaros, Core-Binding family members (RUNX1 and CBF-β), EKLF and E2A (Tenen, et al. (1997) Blood 90, 489-519). In leukemia, many of these transcription factors are involved in chromosomal translocations. TEL (Translocation Ets Leukemia or ETV6), a member of the Ets (E26-transforming-specific) family of transcription factors, is frequently translocated to a variety of partners, resulting in leukemias of both the myeloid and lymphoid compartments. TEL was initially cloned as a translocation partner with the platelet-derived growth factor receptor-β (PDGFRβ) from a patient with chronic myelomonocytic leukemia (Golub, et al. (1994) Cell 77, 307-16). Additionally, TEL is translocated with other tyrosine kinase receptors (TRKC) or other tyrosine kinase recruited to receptors (ABL, JAK2 and ARG), known or presumed transcription factors (AML 1, ARNT, MN 1) in myeloid, lymphoid, chronic and acute leukemias as well as in congenital fibrosarcoma (Papadopoulos, et al. (1995) Cancer Res 55, 34-8; Peeters, et al. (1997) Blood 90, 2535-40; 5. Lacronique, et al. (1997) Science 278, 1309-12; Knezevich, et al. (1998) Cancer Res 58, 5046-8; Knezevich, et al. (1998) Nat Genet 18, 184-7; Eguchi, et al. (1999) Blood 93, 1355-63; Iijima, et al (2000) Blood 95, 2126-31; Romana, et al. (1995) Blood 85, 3662-70; Romana, et al. (1995) Blood 86, 4263-9; Golub, et al. (1995) Proc Natl Acad Sci USA 92, 4917-21; Shurtleff, et al. (1995) Leukemia 9, 1985-9; Salomon-Nguyen, et al. (2000) Proc Natl Acad Sci USA 97, 6757-62). Interestingly, TEL is lost in most cases of TEL/AML1 positive pre-B cell ALL (Stegmaier, et al. (1995) Blood 86, 38-44). This observation suggests that TEL may function as a tumor suppressor.

During fetal development, TEL is required for yolk sac angiogenesis but not for vasculogenesis (Wang, et al. (1997) EMBO J 16, 4374-83). In addition, while primitive (yolk-sac) hematopoiesis does not require TEL chimeric mice generated with TEL ^{-/-} ES cells show that TEL is specifically required for bone marrow hematopoiesis (Wang, et al. (1998) Genes Dev 12, 2392-402). Furthermore, TEL is required for efficient lymphopoiesis in the adult mouse (Wang, et al. (1998) Genes Dev 12, 2392-402). Using chimeras in which TEL ^{+/-} or TEL ^{-/-} ES cells were introduced into RAG-2^{-/-} blastocysts, it was found that TEL is essential for maintaining a pool of lymphoid progenitors in the bone marrow. These data imply that TEL regulates critical target genes at precise stages of hematopoietic development.

TEL contains an 85 amino acid ETS-domain and it has been reported to act as sequence-specific transcriptional repressor on both model and natural promoters (Lopez, et al. (1999) J Biol Chem 274, 30132-8). Furthermore, TEL has been reported to interact via its protein interaction (Pointed or SAM) domain and a central region to a repression complex, which includes SMRT and mSin3A (Chakrabarti, et al. (1999) Biochem Biophys Res Commun 264, 871-7). However, only stromelysin-1 has been identified as a potential target of TEL and it is not yet clear whether this represents a direct or indirect target (Fenrick, et al. (2000) Mol Cell Biol 20, 5828-5839). The identity of additional TEL target genes was sought in an unbiased approach by identifying differentially expressed genes in 32D cells over-expressing TEL.

Transcriptional profiling was performed using oligonucleotide microarrays containing probes for 12000 genes to compare the gene expression profile of the myeloid cell line U937 to U937 cells with inducible TEL expression. Microarray experiments were performed across a time series for TEL induction and the expression of ICSBP was significantly repressed with time. Additionally, global expression profiling of both 32D and 32D/TEL (in which TEL is constitutively expressed) cells over a 24 hr time-course with interferon-gamma (IFN-γ) showed that TEL affects the expression of about 200 genes including ICSBP and the cytostatic effect of IFN-γ is significantly abrogated (˜60%). These data ascribe a previously unassigned role for TEL in IFN-γ signaling and have implications for the role of TEL in the establishment of bone marrow hematopoiesis.

With the use of oligonucleotide arrays (Lockhart, et al. (1996) Nat Biotechnol 14, 1675-80), TEL-mediated repression of the Id1 gene (Lockhart, et al. (1996) Nat Biotechnol 14, 1675-80; Benezra, et al (1990) Cell 61, 49-59) was identified. Furthermore, in vivo binding of TEL to a previously unrecognized Ets binding site within an Id1 enhancer element was demonstrated. Four genes are identified in the present invention to be regulated by TEL, including calcyclin, IL-6, an EST (similar to yeast, YER036C) and Id1. The expression of both calcyclin and IL-6 were elevated, whereas that of the EST similar to yeast YER036C and Id1 were repressed by TEL.

The temporal regulation of Id1 expression is crucial in muscle, neuronal, myeloid and B-cell differentiation programs. In B cell development, both Id1 and Id2 are expressed in pro-B cells and are down regulated following differentiation. Transgenic mice that constitutively over-express Id1 at all stages of B lymphocyte differentiation have impaired B cell development (Sun (1994) Cell 79, 893-900). The arrest in differentiation is at a very early stage and is dosage dependent. The mice have significantly reduced numbers of mature and pre-B cells in the bone marrow, with an associated low frequency of V(D)J and VₖJₖ recombination at the immunoglobulin locus. Interestingly chimeras generated from TEL^{-/-} ES in RAG-2^{-/-} show a dramatic reduction in the frequency and absolute number of TEL^{-/-} B220⁺ B cells in the bone marrow (Wang, et al. (1998) Genes Dev 12, 2392-402). This B cell defect is also observed at the progenitor level, as in the case of the Id1 transgenic animals.

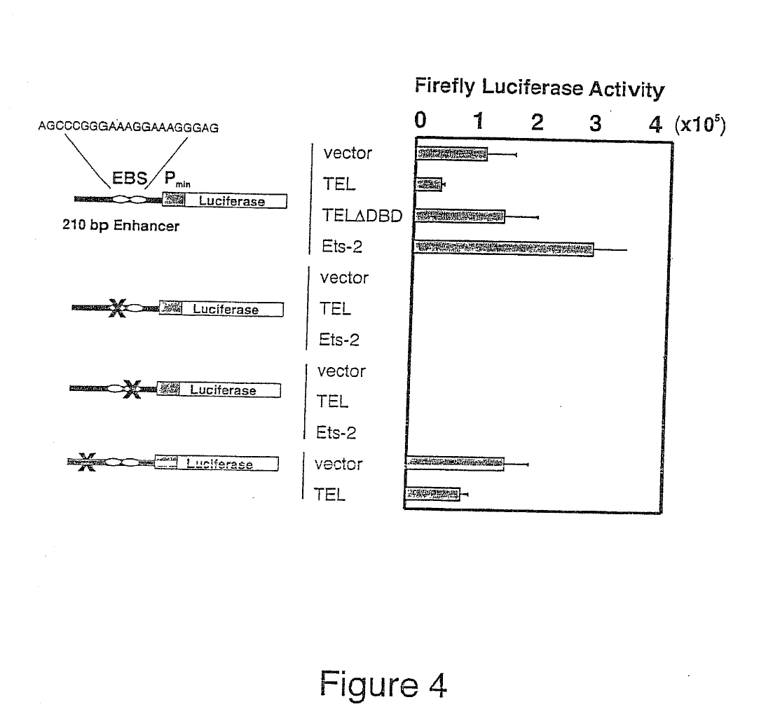

Earlier studies have revealed that C/EBPβ regulates pro-B cell specific expression of Id1 via a 3′ pro-B cell specific enhancer (PBE) (Saisanit and Sun (1997) Mol Cell Biol 15, 1513-1521). Additionally, CHOP, an inhibitor of C/EBPα and β specifically inhibits the formation of C/EBP complexes at the PBE. Regulation, including repression, of Id1 expression has been identified at both the 5′ and 3′ flanking regions of the Id1 gene (Toumay and Benezra (1996) Mol Cell Biol 16, 2418-30). TEL specifically binds to and represses Id1 expression from the EBS within an enhancer region located in the 5′ end of the gene. Unexpectedly, the EBS was absolutely required for basal activity of the enhancer and suggests that Ets protein(s) may play a role in positively regulating Id1 expression. For example, Ets-2 can bind to the Id1 210 bp enhancer region and moreover transactivates the activity of the enhancer in luciferase reporter assays (FIG. 4). Whether it is Ets-2 or another Ets factor that positively regulates Id1 remains to be determined. Support for the concept of opposing effects of Ets family members comes from Drosophila photoreceptor development which is mediated by the Ets proteins YAN and PntP2 (Rebay and Rubin, G. M. (1995) Cell 81, 857-66). YAN is most homologous to TEL in both the DBD and protein interaction domain and like TEL, YAN also acts as a transcriptional repressor.

In pediatric TEL/AML-1 B cell ALL, both alleles of TEL are mutated and result in loss of TEL function (Stegmaier, et al. (1995) Blood 86, 38-44). It is conceivable that loss of TEL function is associated with deregulation of Id1, which may prevent the terminal differentiation of mature B cells. Investigations of Id1 expression in TEL/AML-1 positive and negative pre-B ALL revealed no significant difference in the expression of Id1. This observation may be explained by the fact that in these leukemias, both TEL/AML-1 positive and negative, the B cells are arrested at the pre-B cell stage. Given that in these leukemias, the cells are all at the pre-B cell stage, there would be no significant difference in Id1 and this may be attained through different mechanisms, one of which is loss of TEL function.

AML1/ETO has also been discovered to dysregulate the interferon gamma transcriptional response, by repressing the expression of the interferon regulatory factor ICSBP. It has been demonstrated using gene expression analysis that hematopoietic cells expressing AML1/ETO fail to induce MHC class 2 molecules or the co-stimulatory molecule B7-1 in response to interferon gamma stimulation. This result suggests that one of the roles of these oncogenic fusion proteins is to directly repress the expression of immunomodulatory proteins thereby rendering the transformed cells more capable of escaping immune surveillance. These experiments demonstrate a previously unrecognized level of transcriptional control of the interferon gamma signaling pathway.

Interferon gamma has a cytostatic effect on myeloid cells that is blocked by cells overexpressing either AML1/ETO or TEL, with resulting repression of ICSBP. As shown in the Exemplification, TEL expression represses the activity of the 5′ flanking region of ICSBP (SEQ ID NO: 1), and TEL is associated with the deacetylation of Histone H3. Reintroduction of ICSBP into TEL-overexpressing cells restores the cytostatic effect of interferon gamma, indicating that ICSBP is the critical target of TEL regulation. The leukemogenic transcription factors TEL and AML1/ETO have been discovered, as described herein, to be associated with dysregulation of the interferon gamma transcriptional response, caused by repression of ICSBP expression, which was previously unknown.

The present invention provides a method for diagnosing, aiding in the diagnosing, or predicting, in a subject or individual, a disorder associated with altered leukemogenic transcription factor expression, wherein the leukemogenic transcription factors are TEL or AML1/ETO. The subject or individual for treatment is preferably a mammal, and more preferably a human, however it can be envisaged that any animal with a TEL-related, or AML1/ETO-related, disorder can be treated by the methods of the invention.

The term “target genes” refers to genes that are transcriptionally activated or repressed in response to expression of a leukemogenic transcription factor.

In one embodiment, expression of TEL is associated with repressed expression of target genes ICSBP, Id1 and an EST similar to yeast YER036C. As used herein, repressed expression is considered to be at least about 1.5-fold difference, more preferably at least about 3-fold difference and most preferably at least about 5-fold difference, in comparison with normal expression.

In a further embodiment, expression of TEL is associated with elevated expression of target genes IL-6 or calcyclin. Elevated expression, as used herein, is considered to be at least about 1.5-fold difference, more preferably at least about 3-fold difference and most preferably at least about 5-fold difference, in comparison with normal expression.

As used herein, altered expression of the leukemogenic transcription factor TEL, includes increased expression of TEL, expression of a functional fragment of TEL, expression of TEL fused to another protein as a consequence of a chromosomal translocation event, and loss of TEL expression. Expression of a functional fragment includes expression of a portion or portions of a protein which retain functional activity, such as DNA binding, or interaction with other protein thereby retaining biological activity. TEL fusion proteins can be the result of the TEL gene translocated to, for example, but not limited to, the following genes: PDGFRβ, TRKC, ABL, JAK2, ARG, AML1, ARNT, or MN 1.

In another embodiment, expression of AML1/ETO is associated with the repressed expression of target genes ICSBP, MHC class 2 and B7-1.

Determination of gene expression levels will be clear to one of ordinary skill using techniques that are already established in the art, which include, but are not limited to, analysis of mRNA expression in a sample obtained from a subject being tested using standard techniques such as Northern blot analysis, S1 nuclease analysis, RT-PCR, and gene chip arrays, or analysis of protein expression levels using standard techniques such as SDS-PAGE and western blotting or ELISA techniques, as is well known in the art.

In one embodiment, the disorder associated with altered leukemogenic transcription factor expression is lymphoid, myeloid, acute or chronic leukemia.

The invention also provides for antagonists of the leukemogenic transcription factors or their target genes, as described herein. Generally, the use of antagonists include, and without limitation, methods to modulate leukemogenic transcription factor expression and/or their targets, particularly in a cell, organ, or whole animal. Antagonists can be of any suitable composition, as will be understood by one of skill in the art, and as described herein. For example, antagonists can be an antibody, an anti-sense oligonucleotide, small molecule drug, ribozyme, and the like.

The present invention further relates to antibodies that specifically bind a polypeptide, preferably an epitope, of the present invention (as determined, for example, by immunoassays, a technique well known in the art for assaying specific antibody-antigen binding). Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, for example, anti-Id antibodies to antibodies of the invention), and epitope-binding fragments of any of the above.

The term “antibody,” as used herein, refers to immunoglobulin molecules and immunologically active portions of immunoglobulin molecules, and more specifically, molecules that contain an antigen binding site that specifically binds an antigen. The immunoglobulin molecules of the invention can be of any type (for example, IgG, IgE, IgM, IgD, IgA and IgY), and of any class (for example, IgG1, IgG2, IgG3, IgG4, IgA1 and IgA2) or subclass of an immunoglobulin molecule.

In one embodiment, the antibodies are antigen-binding antibody fragments and include, without limitation, Fab, Fab′ and F(ab′)₂, Fd, single-chain Fvs (scFv), single-chain antibodies, disulfide-linked Fvs (sdFv) and fragments comprising either a V_{L} or V_{H} domain. Antigen-binding antibody fragments, including single-chain antibodies, can comprise the variable region(s) alone or in combination with the entirety or a portion of one or more of the following: hinge region, CH1, CH2, and CH3 domains. Also included in the invention are antigen-binding fragments also comprising any combination of variable region(s) with a hinge region, CH1, CH2, and/or CH3 domains.

The antibodies of the invention may be from any animal origin including birds and mammals. Preferably, the antibodies are human, murine, donkey, sheep, rabbit, goat, guinea pig, hamster, horse, or chicken.

As used herein, “human” antibodies include antibodies having the amino acid sequence of a human immunoglobulin and include antibodies produced by human B cells, or isolated from human sera, human immunoglobulin libraries or from animals transgenic for one or more human immunoglobulins and that do not express endogenous immunoglobulins, as described in U.S. Pat. No. 5,939,598 by Kucherlapati et al., for example.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material.

Antibodies of the present invention may be described or specified in terms of the epitope(s) or portion(s) of a polypeptide of the present invention that they recognize or specifically bind. The epitope(s) or polypeptide portion(s) may be specified, for example, by N-terminal and/or C-terminal positions, or by size in contiguous amino acid residues. Antibodies that specifically bind any epitope or polypeptide of the present invention may also be excluded. Therefore, the present invention includes antibodies that specifically bind polypeptides of the present invention, and allows for the exclusion of the same.

The term “epitope,” as used herein, refers to a portion of a polypeptide which contacts an antigen-binding site(s) of an antibody or T cell receptor. Specific binding of an antibody to an antigen having one or more epitopes excludes non-specific binding to unrelated antigens, but does not necessarily exclude cross-reactivity with other antigens with similar epitopes.

Antibodies of the present invention may also be described or specified in terms of their cross-reactivity. Antibodies of the present invention may not display any cross-reactivity, such that they do not bind any other analog, ortholog, or homolog of a polypeptide of the present invention. Alternatively, antibodies of the invention can bind polypeptides with at least about 95%, 90%, 85%, 80%, 75%, 70%, 65%, 60%, 55%, or 50% identity (as calculated using methods known in the art) to a polypeptide of the present invention. Further included in the present invention are antibodies that bind polypeptides encoded by polynucleotides which hybridize to a polynucleotide of the present invention under stringent hybridization conditions, as will be appreciated by one of skill in the art.

Antibodies of the present invention can also be described or specified in terms of their binding affinity to a polypeptide of the invention. Preferred binding affinities include those with a dissociation constant or Kd less than 5×10⁻⁶ M, 10⁻⁶ M, 5×10⁻⁷ M, 10⁻⁷ M, 5×10⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹³ M, 5×10⁻¹⁵ M, and 10⁻¹⁵ M.

The invention also provides antibodies that competitively inhibit binding of a ligand or antibody to an epitope or epitopes of a polypeptide of the invention, as determined by any method known in the art for determining competitive binding, for example, using immunoassays. In particular embodiments, the antibody competitively inhibits binding to the epitope by at least about 90%, 80%, 70%, 60%, or 50%.

Antibodies of the present invention can act as agonists or antagonists of the polypeptides of the present invention. For example, the present invention includes antibodies which disrupt interactions with the polypeptides of the invention either partially or filly. The invention also includes antibodies that do not prevent binding, but prevent activation or activity of the polypeptide. Activation or activity (for example, signaling) may be determined by techniques known in the art. Also included are antibodies which prevent both binding to and activity of a polypeptide of the invention. Likewise included are neutralizing antibodies.

Antibodies of the present invention may be used, for example, and without limitation, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, for example, Harlow et al., Antibodies: A Laboratory Manual (Cold Spring Harbor Laboratory Press, 2nd ed. 1988).

As discussed in more detail below, the antibodies of the present invention may be used either alone or in combination with other compositions. The antibodies may further be recombinantly fused to a heterologous polypeptide at the N- and/or C-terminus or chemically conjugated (including covalent and non-covalent conjugations) to polypeptides or other compositions. For example, antibodies of the present invention may be recombinantly fused or conjugated to molecules useful as labels in detection assays, or effector molecules such as heterologous polypeptides, drugs, or toxins, as described herein, for example.

The antibodies of the invention include derivatives that are modified, for example, by the covalent attachment of any type of molecule to the antibody such that covalent attachment does not prevent the antibody from recognizing its epitope. For example, but not by way of limitation, the antibody derivatives include antibodies that have been modified, for example, by glycosylation, acetylation, pegylation, phosphorylation, amidation, derivatization by known protecting/blocking groups, proteolytic cleavage, or linkage to a cellular ligand or other protein. Any of numerous chemical modifications can be carried out by known techniques, including, but not limited to, specific chemical cleavage, acetylation, formylation, and metabolic synthesis of tunicamycin. Additionally, the derivative can contain one or more non-classical amino acids.

The antibodies of the present invention can be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, or the like, to induce the production of sera containing polyclonal antibodies specific for the antigen. Various adjuvants can be used to increase the immunological response, depending on the host species, and include, but are not limited to, Freund's adjuvant (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and corynebacterium parvum. Such adjuvants are well known in the art.

Monoclonal antibodies can be prepared using a wide variety of techniques also known in the art, including hybridoma cell culture, recombinant, and phage display technologies, or a combination thereof. For example, monoclonal antibodies can be produced using hybridoma techniques as is known in the art and taught, for example, in Harlow et al., Antibodies: A Laboratory Manual (Cold Spring Harbor Laboratory Press, 2nd ed. 1988). The term “monoclonal antibody” as used herein is not necessarily limited to antibodies produced through hybridoma technology, but also refers to an antibody that is derived from a single clone, including any eukaryotic, prokaryotic, or phage clone.

Human antibodies are desirable for therapeutic treatment of human patients. These antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes. The transgenic mice are immunized with a selected antigen, for example, all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For a detailed discussion of this technology for producing human antibodies and human monoclonal antibodies and protocols for producing such antibodies, see, for example, PCT publications WO 98/24893; WO 96/34096; WO 96/33735; and U.S. Pat. Nos. 5,413,923; 5,625,126; 5,633,425; 5,569,825; 5,661,016; 5,545,806; 5,814,318; and 5,939,598.

In another embodiment, antibodies to the polypeptides of the invention can, in turn, be utilized to generate anti-idiotype antibodies that “mimic” polypeptides of the invention using techniques well known to those skilled in the art. (See, for example, Greenspan & Bona (1989) FASEB J. 7(5):437-444 and Nissinoff, (1991) J. Immunol. 147(8):2429-2438). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize a polypeptide ligand. For example, such anti-idiotypic antibodies can be used to bind a polypeptide of the invention and/or to bind its ligands, and thereby block its biological activity.

The antibodies or fragments thereof of the present invention can be fused to marker sequences, such as a peptide to facilitate their purification. In one embodiment, the marker amino acid sequence is a hexa-histidine peptide, an HA tag, or a FLAG tag, all of which are commercially available and appreciated by one of skill in the art.

The present invention further encompasses antibodies or fragments thereof conjugated to a diagnostic or therapeutic agent. The antibodies can be used diagnostically, for example, to monitor the development or progression of a tumor as part of a clinical testing procedure to determine the efficacy of a given treatment regimen. Detection can be facilitated by coupling the antibody to a detectable substance. Examples of detectable substances include enzymes (such as, horseradish peroxidase, alkaline phosphatase, beta-galactosidase, or acetylcholinesterase), prosthetic group (such as streptavidin/biotin and avidin/biotin), fluorescent materials (such as umbelliferone, fluorescein, fluorescein isothiocyanate, rhodamine, dichlorotriazinylamine fluorescein, dansyl chloride or phycoerythrin), luminescent materials (such as luminol), bioluminescent materials (such as luciferase, luciferin, and aequorin), radioactive materials (such as, ¹²⁵I, ¹³¹I, ¹¹¹In or ⁹⁹Tc), and positron emitting metals using various positron emission tomographies, and nonradioactive paramagnetic metal ions.

In an additional embodiment, an antibody or fragment thereof can be conjugated to a therapeutic moiety such as a cytotoxin, for example, a cytostatic or cytocidal agent, a therapeutic agent or a radioactive metal ion. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (for example, daunorubicin (formerly daunomycin) and doxorubicin), antibiotics (for example, actinomycin, bleomycin, mithramycin, and anthramycin (AMC)), and anti-mitotic agents (for example, vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, a thrombotic agent or an anti-angiogenic agent, for example, angiostatin or endostatin; or, biological response modifiers such as, for example, lymphokines, interleukins, granulocyte macrophase colony stimulating factor (“GM-CSF”), granulocyte colony stimulating factor (“G-CSF”), or other growth factors.

Antibodies of the invention can also be attached to solid supports. These are particularly useful for immunoassays or purification of the target antigen. Such solid supports include, but are not limited to, glass, cellulose, silicon, polyacrylamide, nylon, polystyrene, polyvinyl chloride or polypropylene. Techniques for conjugating such therapeutic moiety to antibodies are well known in the art, see, for example, Arnon et al., “Monoclonal Antibodies For Immunotargeting Of Drugs In Cancer Therapy”, in Monoclonal Antibodies And Cancer Therapy, Reisfeld et al eds., pp. 243-56 (Alan R. Liss, Inc. 1985).

Alternatively, an antibody can be conjugated to a second antibody to form an antibody heteroconjugate as described by Segal in U.S. Pat. No. 4,676,980.

An antibody of the invention, with or without conjugation to a therapeutic moiety, administered alone or in combination with cytotoxic factor(s) and/or cytokine(s), can be used as a therapeutic.

Antisense antagonists of the present invention are also included. Antisense technology can be used to control gene expression through antisense DNA or RNA, or through triple-helix formation. Antisense techniques are discussed for example, in Okano (1991) Neurochem. 56:560. The methods are based on binding of a polynucleotide to a complementary DNA or RNA. In one embodiment, an antisense sequence is generated internally by the organism, in another embodiment, the antisense sequence is separately administered (see, for example, O'Connor (1991) Neurochem. 56:560).

In one embodiment, the 5′ coding portion of a polynucleotide that encodes a polypeptide of the present invention can be used to design an antisense RNA oligonucleotide from about 10 to 40 base pairs in length. Generally, a DNA oligonucleotide is designed to be complementary to a region of the gene involved in transcription thereby preventing transcription and the production of the receptor. The antisense RNA oligonucleotide hybridizes to the mRNA in vivo and blocks translation of the mRNA molecule into receptor polypeptide.

In one embodiment, the antisense nucleic acid of the invention is produced intracellularly by transcription from an exogenous sequence. For example, a vector or a portion thereof, is transcribed, producing an antisense nucleic acid of the invention. Such a vector contains the sequence encoding the antisense nucleic acid. The vector can remain episomal or become chromosomally integrated, as long as it can be transcribed to produce the desired antisense RNA. Vectors can be constructed by recombinant DNA technology and can be plasmid, viral, or otherwise, as is known to one of skill in the art.

Expression can be controlled by any promoter known in the art to act in the target cells, such as vertebrate cells, and preferably human cells. Such promoters can be inducible or constitutive and include, without limitation, the SV40 early promoter region (Bemoist and Chambon (1981) Nature 29:304-310), the promoter contained in the 3′ long terminal repeat of Rous sarcoma virus (Yamamoto et al. (1980) Cell 22:787-797), the herpes thymidine promoter (Wagner et al. (1981) Proc. Natl. Acad. Sci. U.S.A. 78:1441-1445), and the regulatory sequences of the metallothionein gene (Brinster, et al. (1982) Nature 296:39-42).

The antisense nucleic acids of the invention comprise a sequence complementary to at least a portion of an RNA transcript of a gene of the invention. Absolute complementarity, although preferred, is not required. A sequence “complementary to at least a portion of an RNA,” referred to herein, means a sequence having sufficient complementarity to be able to hybridize with the RNA, forming a stable duplex. The ability to hybridize will depend on both the degree of complementarity and the length of the antisense nucleic acid. Generally, the larger the hybridizing nucleic acid, the more base mismatches with the RNA it may contain and still form a stable duplex. One skilled in the art can ascertain a tolerable degree of mismatch by use of standard procedures to determine the melting point of the hybridized complex.

Oligonucleotides that are complementary to the 5′ end of the RNA, for example, the 5′ untranslated sequence up to and including the AUG initiation codon, are generally regarded to work most efficiently at inhibiting translation. However, sequences complementary to the 3′ untranslated sequences of mRNAs have been shown to be effective at inhibiting translation of mRNAs as well. Thus, oligonucleotides complementary to either the 5′- or 3′-non-translated, non-coding regions of a nucleotide sequence can be used in an antisense approach to inhibit mRNA translation. Oligonucleotides complementary to the 5′ untranslated region of the mRNA can include the complement of the AUG start codon. Antisense oligonucleotides complementary to mRNA coding regions can also be used in accordance with the invention. In one embodiment, the antisense nucleic acids are at least six nucleotides in length, and are preferably oligonucleotides ranging from about 6 to about 50 nucleotides in length. In other embodiments, the oligonucleotide is at least about 10, 17, 25 or 50 nucleotides in length.

The polynucleotides of the invention can be DNA or RNA, or chimeric mixtures, or derivatives or modified versions thereof, single-stranded or double-stranded. The oligonucleotide can be modified at the base moiety, sugar moiety, or phosphate backbone, for example, to improve stability of the molecule, hybridization, and the like. The oligonucleotide can include other appended groups such as peptides (for example, to target host cell receptors in vivo), or agents that facilitate transport across the cell membrane, or the blood-brain barrier, or intercalating agents.

The antisense oligonucleotide may comprise at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, a-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An a-anomeric oligonucleotide forms specific double-stranded hybrids with complementary RNA in which, contrary to the usual b-units, the strands run parallel to each other (Gautier et al. (1987) Nuc. Acids Res. 15:6625-6641). The oligonucleotide is a 2′-O-methylribonucleotide (Inoue et al.,(1987) Nuc. Acids Res. 15:6131-6148), or a chimeric RNA-DNA analog (Inoue et al., (1987) FEBS Lett. 215:327-330).

Polynucleotides of the invention may be synthesized by standard methods known in the art, for example, by use of an automated DNA synthesizer.

Potential antagonists according to the invention also include catalytic RNA, or a ribozyme. Hammerhead ribozymes cleave mRNAs at locations dictated by flanking regions that form complementary base pairs with the target mRNA. The target mRNA has the following sequence of two bases: 5′-UG-3′. The construction and production of hammerhead ribozymes is well known in the art and is described more fully in Haseloff and Gerlach, (1988) Nature 334:585-591. Preferably, the ribozyme is engineered so that the cleavage recognition site is located near the 5′ end of the mRNA in order to increase efficiency and minimize the intracellular accumulation of non-functional mRNA transcripts.

Ribozymes of the invention can be composed of modified oligonucleotides (for example for improved stability, targeting, and the like). DNA constructs encoding the ribozyme can be under the control of a strong constitutive promoter, such as, for example, pol III or pol II promoter, so that a transfected cell will produce sufficient quantities of the ribozyme to destroy endogenous target mRNA and inhibit translation. Since ribozymes, unlike antisense molecules, are catalytic, a lower intracellular concentration is generally required for efficiency.

Another embodiment of the invention are methods to screen for agents or compounds (also referred to herein as “screening assays”) that are agonists, mimics or antagonists of the subject leukemogenic transcription factors. The term “agonist” refers to agents that potentiate or stimulate the activities of the leukemogenic factor(s) or their target genes, the term “mimic” refers to agents that cause effects in the same manner as the leukemogenic factor(s) or target genes, and the term “antagonist” refers to agenet that oppose or interfere with the activities of the leukemogenic factor(s) or target genes. Agonists, mimics or antagonists can be from any natural or synthetic source, and include for example and without limitation, polypeptides, such as antibodies, fusion proteins, small molecule drugs, peptidomimetics, prodrugs, receptors, binding agents, intercalating agents, oligonucleotides such as anti-sense oligonucleotides, ribozymes and the like.

Such agents or compounds can bind to the genes or their encoded polypeptide products of the invention (e.g., TEL, AML1/ETO, ICSBP, Id1, EST similar to YER036C, IL-6, calcylcin, B7-1 or MHC class 2), and have a stimulatory or inhibitory effect. Additionally or alternatively, said agents or compounds can change or modulate (e.g., enhance or inhibit) the ability of the genes or their encoded polypeptide products of the invention to interact with other compounds or agents that bind the genes or their encoded polypeptide products of the invention. Furthermore, said agents or compounds can alter post-translational processing of such the genes or their encoded polypeptide products of the invention (e.g., agents that alter proteolytic processing to direct the gene expression product or the encoded polypeptide products from where it is normally synthesized to another location in the cell, such as the cell surface, a specific organelle, or the nucleus; or agents that alter proteolytic processing such that a gene expression product or the encoded polypeptide product described herein is secreted or released from the cell, etc.).

The agent or compound can cause an increase in the activity of a leukemogenic transcription factor of the present invention, or target thereof. For example, the activity can be increased by at least 1.5-fold to 2-fold, at least 3-fold, or, at least 5-fold, relative to the control. Alternatively, the activity can be decreased, for example, by at least 10%, at least 20%, 40%, 50%, or 75%, or by at least 90%, relative to a suitable control.

In one embodiment, the invention provides assays for screening agents or compounds to identify agents or compounds that bind to, or modulate the activity of, a leukemogenic transcription factor described herein, or target thereof (including biologically active portion(s) thereof), as well as the agents identified by the assays. As used herein, a “compound” or “agent” is a chemical molecule, be it naturally-occurring or artificially-derived, and includes, for example, peptides, proteins, synthesized molecules, for example, synthetic organic molecules, naturally-occurring molecule, for example, naturally occurring organic molecules, nucleic acid molecules, and components thereof.

In general, agents or compounds for use in the present invention may be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. For example, candidate agents or compounds can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide libraries, while the other four approaches are applicable to polypeptide, non-peptide oligomer or small molecule libraries of compounds (Lam, Anticancer Drug Des., 12: 145 (1997)). Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activities should be employed whenever possible.

When a crude extract is found to modulate (i.e., stimulate or inhibit) the expression and/or activity of a leukemogenic transcription factor, or target thereof, and/or their encoded polypeptides, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits nucleic acid expression, polypeptide expression, or polypeptide biological activity. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases in which it is desirable to alter the activity or expression of the nucleic acids or polypeptides of the present invention.

In one embodiment, to identify agents or compounds (also referred to herein as “candidate agents or candidate compounds”), that alter the biological activity of a polypeptide encoded by a leukemogenic transcription factor, or target thereof, as described herein, a cell, tissue, cell lysate, tissue lysate, or solution containing or expressing a polypeptide encoded by the leukemogenic transcription factor, or target thereof (e.g., a polypeptide encoded by TEL, AML1/ETO, ICSBP, Id1, EST similar to YER036C, IL-6, calcylcin, B7-1 or MHC class 2), or a fragment or derivative thereof, can be contacted with an agent or compound to be tested under conditions suitable for biological activity of the polypeptide. Alternatively, the polypeptide can be contacted directly with the agent or compound to be tested. The level (amount) of polypeptide biological activity is assessed/measured, either directly or indirectly, and is compared with the level of biological activity in a control (i.e., the level of activity of the polypeptide or active fragment or derivative thereof in the absence of the agent or compound to be tested, or in the presence of the agent or compound vehicle only). If the level of the biological activity in the presence of the agent or compound differs, by an amount that is statistically significant, from the level of the biological activity in the absence of the agent or compound, or in the presence of the agent or compound vehicle only, then the agent or compound is an agent or compound that alters the biological activity of the polypeptide encoded by a leukemogenic transcription factor of the invention, or target thereof. Typically, an increase in the level of polypeptide biological activity relative to a control, indicates that the agent or compound enhances (is an agonist of) the polypeptide biological activity. Similarly, a decrease in the polypeptide biological activity relative to a control, indicates that the agent or compound inhibits (is an antagonist of) the polypeptide biological activity.

In another embodiment, the level of biological activity of a polypeptide encoded by a leukemogenic transcription factor, or target thereof, or a derivative or fragment thereof, in the presence of the agent or compound to be tested, is compared with a control level that has previously been established. A level of polypeptide biological activity in the presence of the agent or compound that differs from (i.e., increases or decreases) the control level by an amount that is statistically significant indicates that the agent or compound alters the biological activity of the polypeptide.

The present invention also relates to an assay for identifying agents or compounds (e.g., antisense nucleic acids, fusion proteins, polypeptides, peptidomimetics, prodrugs, receptors, binding agents, antibodies, small molecules or other drugs, or ribozymes) that alter (e.g., increase or decrease) expression (e.g., transcription or translation) of a leukemogenic transcription factor, or target thereof, or that otherwise interacts with a leukemogenic transcription factor, or target thereof, as described herein, as well as compounds identifiable by the assays. For example, a solution containing a leukemogenic transcription factor, or target thereof, can be contacted with an agent or compound to be tested. The solution can comprise, for example, cells containing the leukemogenic transcription factor, or target thereof, or cell lysate containing the leukemogenic transcription factor, or target thereof, alternatively, the solution can be another solution that comprises elements necessary for transcription/translation of the leukemogenic transcription factor, or target thereof. Cells not suspended in solution can also be employed, if desired. The level and/or pattern of leukemogenic transcription factor expression, or target thereof, (e.g., the level and/or pattern of mRNA or protein expressed) is assessed, and is compared with the level and/or pattern of expression in a control (i.e., the level and/or pattern of the leukemogenic transcription factor, or target thereof, expressed in the absence of the agent or compound, or in the presence of the agent or compound vehicle only). If the expression level and/or pattern in the presence of the agent or compound differs by an amount or in a manner that is statistically significant from the level and/or pattern in the absence of the agent or compound, or in the presence of the agent or compound vehicle only, then the agent or compound is an agent or compound that alters the expression of a leukemogenic transcription factor, or target thereof.

In another embodiment, the level and/or pattern of a leukemogenic transcription factor, or target thereof, in the presence of the agent or compound to be tested, is compared with a control level and/or pattern that has previously been established. A level and/or pattern leukemogenic transcription factor, or target thereof, expression in the presence of the agent or compound that differs from the control level and/or pattern by an amount or in a manner that is statistically significant indicates that the agent or compound alters leukemogenic transcription factor expression, or target thereof.

In another embodiment of the invention, compounds that alter the expression of a leukemogenic transcription factor, or target thereof, or that otherwise interact with a leukemogenic transcription factor, or target thereof, as described herein, can be identified using a cell, cell lysate, or solution containing a nucleic acid encoding the promoter region of the leukemogenic transcription factor, or target thereof, operably linked to a reporter gene. As used herein by “promoter” means a minimal nucleotide sequence sufficient to direct transcription, and by “operably linked” means that a gene and one or more regulatory sequences are connected in such a way as to permit gene expression when the appropriate molecules (e.g., transcriptional activator proteins) are bound to the regulatory sequences. Examples of reporter genes and methods for operably linking a reporter gene to a promoter are known in the art. After contact with an agent or compound to be tested, the level of expression of the reporter gene (e.g., the level of mRNA or of protein expressed) is assessed, and is compared with the level of expression in a control (i.e., the level of expression of the reporter gene in the absence of the agent or compound, or in the presence of the agent or compound vehicle only). If the level of expression in the presence of the agent or compound differs by an amount or in a manner that is statistically significant from the level in the absence of the agent or compound, or in the presence of the agent or compound vehicle only, then the agent or compound alters the expression of the leukemogenic transcription factor, or target thereof, as indicated by its ability to alter expression of the reporter gene that is operably linked to the leukemogenic transcription factor, or target thereof.

In another embodiment, the level of expression of the reporter in the presence of the agent or compound to be tested, is compared with a control level that has previously been established. A level in the presence of the agent or compound that differs from the control level by an amount or in a manner that is statistically significant indicates that the agent or compound alters leukemogenic transcription factor, or target thereof, expression.

The present invention also features methods of detecting and/or identifying an agent or compound that alters the interaction between a polypeptide encoded by a leukemogenic transcription factor, or target thereof, and a polypeptide (or other molecule) with which the leukemogenic transcription factor polypeptide, or target thereof, normally interacts with (e.g., in a cell or under physiological conditions). In one example, a cell or tissue that expresses or contains a compound (e.g., a polypeptide or other molecule, wherein such a molecule is referred to herein as a “polypeptide substrate”) that interacts with a polypeptide encoded by a leukemogenic transcription factor, or target thereof, is contacted with the leukemogenic transcription factor polypeptide, or target thereof, in the presence of an agent or compound, and the ability of the agent or compound to alter the interaction between the polypeptide encoded by the leukemogenic transcription factor, or target thereof, and the polypeptide substrate is determined, for example, by assaying activity of the transcription factor polypeptide, or target thereof. Alternatively, a cell lysate or a solution containing the leukemogenic transcription factor polypeptide, or target thereof, the polypeptide substrate, and the agent or compound can be used. An agent or compound that binds to the leukemogenic transcription factor polypeptide, or target thereof, or to the polypeptide substrate, can alter the interaction between the leukemogenic transcription factor polypeptide, or target thereof, and the polypeptide substrate by interfering with (inhibiting), or enhancing (stimulating) the ability of the leukemogenic transcription factor, or target thereof, to bind to, associate with, or otherwise interact with the polypeptide substrate.

Determining the ability of the agent or compound to bind to the leukemogenic transcription factor, or target thereof or a polypeptide substrate can be accomplished, for example, by coupling the agent or compound with a radioisotope or enzymatic label such that binding of the agent or compound to the leukemogenic transcription factor polypeptide, or target thereof, or polypeptide substrate can be determined by directly or indirectly detecting the agent or compound labeled with ¹²⁵I, ³⁵S, ¹⁴C, or ³H, and the detecting the radioisotope (e.g., by direct counting of radioemmission or by scintillation counting). Alternatively, the agent or compound can be enzymatically labeled with, for example, horseradish peroxidase, alkaline phosphatase, or luciferase, and the enzymatic label is then detected by determination of conversion of an appropriate substrate to product. In another alternative, one of the other components of the screening assay (e.g., the polypeptide substrate, the leukemogenic transcription factor, or target thereof) can be labeled, and alterations in the interaction between the leukemogenic transcription factor, or target thereof, and the polypeptide substrate can be detected. In these methods, labeled unbound components can be removed (e.g., by washing) after the interaction step in order to accurately detect the effect of the agent or compound on the interaction between the leukemogenic transcription factor, or target thereof, and the polypeptide substrate.

It is also within the scope of this invention to determine the ability of an agent or compound to interact with the leukemogenic transcription factor, or target thereof, or polypeptide substrate without the labeling of any of the interactants. For example, a microphysiometer can be used to detect the interaction of an agent or compound with a polypeptide encoded by an leukemogenic transcription factor, or target thereof, or a polypeptide substrate without the labeling of either the agent or compound, the polypeptide encoded by the leukemogenic transcription factor, or target thereof, or the polypeptide substrate (McConnell et al., (1992) Science, 257: 1906-1912). As used herein, a “microphysiometer” (e.g., CYTOSENSOR™) is an analytical instrument that measures the rate at which a cell acidifies its environment using a light-addressable potentiometric sensor (LAPS). Changes in this acidification rate can be used as an indicator of the interaction between ligand and polypeptide.

In another embodiment of the invention, assays can be used to identify polypeptides that interact with one or more polypeptides encoded by a leukemogenic transcription factor, or target thereof, as described herein. For example, a yeast two-hybrid system such as that described by Fields and Song (Fields and Song, Nature 340: 245-246 (1989)) can be used to identify polypeptides that interact with one or more polypeptides encoded by a leukemogenic transcription factor, or target thereof. In such a yeast two-hybrid system, vectors are constructed based on the flexibility of a transcription factor that has two functional domains (a DNA binding domain and a transcription activation domain). If the two domains are separated but fused to two different proteins that interact with one another, transcriptional activation can be achieved, and transcription of specific markers (e.g., nutritional markers such as His and Ade, or color markers such as lacZ) can be used to identify the presence of interaction and transcriptional activation. For example, in the methods of the invention, a first vector is used that includes a nucleic acid encoding a DNA binding domain and a polypeptide encoded by a leukemogenic transcription factor, or target thereof, or fragment or derivative thereof, and a second vector is used that includes a nucleic acid encoding a transcription activation domain and a nucleic acid encoding a polypeptide that potentially may interact with the leukemogenic transcription factor, or target thereof, or fragment or derivative thereof. Incubation of yeast containing the first vector and the second vector under appropriate conditions (e.g., mating conditions such as used in the MATCHMAKER™ system from Clontech) allows identification of colonies that express the markers of the polypeptide(s). These colonies can be examined to identify the polypeptide(s) that interact with the polypeptide encoded by the leukemogenic transcription factor, or target thereof, or a fragment or derivative thereof. Such polypeptides may be useful as compounds that alter the activity or expression of the leukemogenic transcription factor polypeptide, or target thereof, as described herein.

In more than one embodiment of the above assay methods of the present invention, it may be desirable to immobilize a polypeptide encoded by a leukemogenic transcription factor, or target thereof, or a polypeptide substrate, or other components of the assay on a solid support, in order to facilitate separation of complexed from uncomplexed forms of one or both of the polypeptides, as well as to accommodate automation of the assay. Binding of an agent or compound to the polypeptide, or interaction of the polypeptide with a polypeptide substrate in the presence and absence of an agent or compound, can be accomplished in any vessel suitable for containing the reactants. Examples of such vessels include microtitre plates, test tubes, and micro-centrifuge tubes. In one embodiment, a fusion protein (e.g., a glutathione-S-transferase fusion protein) can be provided that adds a domain that allows the leukemogenic transcription factor polypeptide, or target thereof, or the polypeptide substrate to be bound to a matrix or other solid support.

This invention further pertains to novel agents or compounds identified by the above-described screening assays. Accordingly, it is within the scope of this invention to further use an agent or compound identified as described herein in an appropriate animal model. For example, an agent or compound identified as described herein can be used in an animal model to determine the efficacy, toxicity, or side effects of treatment with such an agent or compound. Alternatively, an agent or compound identified as described herein can be used in an animal model to determine the mechanism of action of such an agent or compound. Furthermore, this invention pertains to uses of novel agents and compounds identified by the above-described screening assays for treatments as described herein. In addition, an agent or compound identified as described herein can be used to alter activity of a polypeptide encoded by an leukemogenic transcription factor, or target thereof, or to alter expression of the leukemogenic transcription factor, or target thereof, by contacting the polypeptide or the nucleic acid molecule (or contacting a cell comprising the polypeptide or the nucleic acid molecule) with the agent or compound identified as described herein.

An additional method to screen for agents that are agonists, mimics or antagonists comprises culturing test cells that express TEL or AML1/ETO in a suitable culture medium as can be readily determined by those skilled in the art. Test cells include, without limitation, hematopoietic cells, bone marrow-derived cells, splenocytes, or circulating lymphocytes, the collection of which from a subject or individual are standard medical procedures known to those of skill in the art. Alternatively, test cells can be a cell line that overexpresses TEL or AML1/ETO. In a preferred embodiment, the cell lines are of mammalian origin. The test cells are exposed to the agent to be tested and the cells are subsequently analyzed for altered expression of target genes. Target gene expression in test cells treated with the agent is compared with target gene expression in suitable control cells, such as cells that do not express altered levels of TEL or AML1/ETO. Alternatively, target gene expression in test cells is compared with basal expression levels of target gene expression in test cells not treated with agent. Repressed expression of ICSBP, Id1 or EST similar to yeast YER036C, or increased expression of IL-6 or calcyclin in TEL expressing cells, as compared with control cells or basal expression levels, is indicative that the agent is an agonist or mimic of TEL activity, whereas increased expression of ICSBP, Id1 and EST similar to yeast YER036C, or repressed expression of IL-6 or calcyclin in TEL expressing cells as compared to control cells or basal expression levels is indicative that the agent is an antagonist of TEL activity. Furthermore, repressed expression of ICSBP, MHC class 2 or B7-1 in AML1/ETO expressing cells, in comparison with control cells or basal expression levels, is indicative that the agent is an agonist or mimic of AML1/ETO activity, whereas elevated expression of ICSBP, MHC class 2 or B7-1 in AML1/ETO expressing cells in comparison with control cells or basal expression levels is indicative that the agent is an antagonist of AML1/ETO activity.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder. Effective doses may be extrapolated from dose-response curves derived from in vitro or animal model test systems.

The compounds or pharmaceutical compositions of the invention can be tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

In another embodiment of this invention, a method for determining the effectiveness of an agent that modulates TEL activity or AML1/ETO activity for treatment of a disorder characterized by altered TEL expression or altered AML1/ETO expression is provided. Effectiveness is determined, for example, as the capacity to affect the proliferation or differentiation status of the test cells. The method comprises obtaining from the subject test cells that have altered TEL expression or altered AML1/ETO expression. Test cells include, without limitation, hematopoietic cells, bone marrow-derived cells, splenocytes, or circulating lymphocytes, the collection of which from a subject or individual are standard medical procedures known to those of skill in the art. Alternatively, test cells can be a cell line that overexpresses TEL or AML1/ETO. In one embodiment, the cell line is of mammalian origin. The test cells are subsequently cultured in a suitable medium in the absence or presence of the agent to be tested, wherein increased differentiation of the test cells in the presence of the agent, relative to test cells in the absence of the agent, is predictive of the efficacy of the agent for the treatment of the disorder. The differentiation status of test cells may be readily achieved using differentiation markers for the cells being tested, as will be apparent to those of ordinary skill in the art.

Alternatively, or in addition, test cells are cultured in a suitable medium in the absence or presence of the agent to be tested, wherein decreased proliferation of the test cells in the presence of the agent, relative to test cells in the absence of the agent, is predictive of the efficacy of the agent for the treatment of the disorder. Proliferation of test cells can be determined using techniques known in the art, which includes but is not limited to, tritiated thymidine uptake, BrdU incorporation, or cell counting techniques.

The present invention also provides pharmaceutical compositions, including both therapeutic and prophylatic compositions. Compositions within the scope of this invention include all compositions wherein the antibody, fragment or derivative, antisense oligonucleotide or ribozyme is contained in an amount effective to achieve its intended purpose. While individual needs vary, determination of optimal ranges of effective amounts of each component is within the skill of the art. The effective dose is a function of a number of factors, including the specific antibody, the antisense construct, ribozyme or polypeptide of the invention, the presence of a conjugated therapeutic agent, the patient and their clinical status.

Such compositions generally comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skimmed milk, glycerol, propylene, glycol, water, ethanol and the like. The composition, if desired, can also contain minor amounts of wetting or emulsifying agents, or pH buffering agents.

These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to a human. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, and the like, and those formed with cations such as those derived from sodium, potassium, ammonium, calcium, ferric hydroxides, isopropylamine, triethylamine, 2-ethylamino ethanol, histidine, procaine, and the like.

The compositions of the invention can be administered alone or in combination with other therapeutic agents. Therapeutic agents that can be administered in combination with the compositions of the invention, include but are not limited to chemotherapeutic agents, antibiotics, steroidal and non-steroidal anti-inflammatories, conventional immunotherapeutic agents, cytokines and/or growth factors. Combinations may be administered either concomitantly, for example, as an admixture, separately but simultaneously or concurrently; or sequentially. This includes presentations in which the combined agents are administered together as a therapeutic mixture, and also procedures in which the combined agents are administered separately but simultaneously, for example, as through separate intravenous lines into the same individual. Administration “in combination” further includes the separate administration of one of the compounds or agents given first, followed by the second.

Conventional nonspecific immunosuppressive agents, that may be administered in combination with the compositions of the invention include, but are not limited to, steroids, cyclosporine, cyclosporine analogs, cyclophosphamide methylprednisone, prednisone, azathioprine, FK-506, 15-deoxyspergualin, and other immunosuppressive agents.

In a further embodiment, the compositions of the invention are administered in combination with an antibiotic agent. Antibiotic agents that may be administered with the compositions of the invention include, but are not limited to, tetracycline, metronidazole, amoxicillin, beta-lactamases, aminoglycosides, macrolides, quinolones, fluoroquinolones, cephalosporins, erythromycin, ciprofloxacin, and streptomycin.

In an additional embodiment, the compositions of the invention are administered alone or in combination with an anti-inflammatory agent. Anti-inflammatory agents that can be administered with the compositions of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In another embodiment, compositions of the invention are administered in combination with a chemotherapeutic agent. Chemotherapeutic agents that may be administered with the compositions of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carnustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In an additional embodiment, the compositions of the invention are administered in combination with cytokines. Cytokines that may be administered with the compositions of the invention include, but are not limited to, IL-2, IL-3, L-4, IL-5, IL-6, IL-7, IL-10, IL-12, IL-13, IL-15, anti-CD40, CD40L, IFN-gamma and TNF-alpha.

In additional embodiments, the compositions of the invention are administered in combination with other therapeutic or prophylactic regimens, such as, for example, radiation therapy.

The present invention is further directed to therapies which involve administering pharmaceutical compositions of the invention to an animal, preferably a mammal, and most preferably a human, patient for treating one or more of the described disorders. Therapeutic compositions of the invention include, for example, antibodies of the invention (including fragments, analogs and derivatives thereof as described herein), antisense oligonucleotides, ribozymes and nucleic acids encoding same. The compositions of the invention can be used to treat, inhibit, prognose, diagnose or prevent diseases, disorders or conditions associated with aberrant expression and/or activity of a polypeptide of the invention, including, but not limited to, any one or more of the diseases, disorders, or conditions such as, for example, a lymphoproliferative disorder, lymphoid leukemia, myeloid leukemia, acute leukemia or chronic leukemia.

The treatment and/or prevention of diseases and disorders associated with aberrant expression and/or activity of a polypeptide of the invention includes, but is not limited to, alleviating symptoms associated with those diseases and disorders.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Furthermore, the dosage and frequency of administration of antibodies of the invention may be reduced by enhancing uptake and tissue penetration of the antibodies by modifications such as, for example, lipidation or addition of cell-specific tags.

The invention provides methods of treatment, inhibition and prophylaxis by administration to a subject of an effective amount of a compound or pharmaceutical composition of the invention. In one aspect, the compound is substantially purified such that the compound is substantially free from substances that limit its effect or produce undesired side-effects. The subject is preferably an animal, including but not limited to animals such as cows, pigs, horses, chickens, cats, dogs, etc., and is preferably a mammal, and most preferably human.

Various delivery systems are known and can be used to administer a composition of the invention, for example, encapsulation in liposomes, microparticles, microcapsules, recombinant cells capable of expressing the compound, receptor-mediated endocytosis (see, e.g., Wu and Wu, (1987) J. Biol. Chem. 262:4429-4432), construction of a nucleic acid as part of a retroviral or other vector, and the like as will be known by one of skill in the art.

Methods of introduction include, but are not limited to, intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, for example, by use of an inhaler or nebulizer, and formulation with an aerosolizing agent.

In one embodiment, it may be desirable to administer the pharmaceutical compounds or compositions of the invention locally to the area in need of treatment; this may be achieved by, for example, and not by way of limitation, local infusion during surgery, topical application, for example, in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, such as a liposome (Langer, (1990) Science 249:1527-1533).

In yet another embodiment, the compound or composition can be delivered in a controlled release system. Furthermore, a controlled release system can be placed in proximity of the therapeutic target, thus requiring only a fraction of the systemic dose (see, e.g., Goodson, (1984) in Medical Applications of Controlled Release, supra, vol. 2, pp. 115-138). In a further embodiment, a pump may be used. In another embodiment, polymeric materials can be used.

In a particular embodiment where the compound of the invention is a nucleic acid encoding a protein, the nucleic acid can be administered in vivo to promote expression of its mRNA and encoded protein, by constructing it as part of an appropriate nucleic acid expression vector and administering, for example, by use of a retroviral vector, or by direct injection, or by use of microparticle bombardment for example, a gene gun, or coating with lipids or cell-surface receptors or transfecting agents, or by administering it in linkage to a homeobox-like peptide which is known to enter the nucleus (see e.g., Joliot el al., (1991) Proc. Natl. Acad Sci. USA 88:1864-1868). Alternatively, a nucleic acid can be introduced intracellularly and incorporated within host cell DNA for expression, by homologous recombination.

A further embodiment of the invention relates to the treatment of an individual having a disorder characterized by altered TEL expression, elevated or repressed expression of any one of ICSBP, Id1, calcyclin, EST similar to yeast YER036C, or IL-6, wherein an effective amount of an agent that modulates TEL activity is administered to the individual. Optionally, the agent can be administered in combination with a pharmaceutical carrier as is commonly used in the art. As described above, administration of the agent may be orally, intravenously, intramuscularly, subcutaneously, topically, rectally, or by inhalation. Administration can be formulated to deliver the agent in a single dosage, multiple dosages, or to gradually infuse over time. Other methods of administration will be known to those skilled in the art. Disorders that may be treated with these agents include lymphoid, myeloid, acute and chronic leukemias. The subject or individual for treatment is preferably a mammal, and more preferably a human, however it can be envisaged that any animal with a TEL-related disorder can be treated by the method of the invention.

Another embodiment of the invention relates to the treatment of an individual having a disorder characterized by AML1/ETO expression, elevated or repressed expression of any one of ICSBP, MHC class 2 or B7-1, wherein an effective amount of an agent that modulates AML1/ETO activity is administered to the individual. Optionally, the agent can be administered in combination with a pharmaceutical carrier as is commonly used in the art. Administration of the agent may be orally, intravenously, intramuscularly, subcutaneously, topically, rectally, or by inhalation. Administration can be formulated to deliver the agent in a single dosage, multiple dosages, or to gradually infuse over time. Other methods of administration will be known to those skilled in the art. Disorders that may be treated with these agents include lymphoid, myeloid, acute and chronic leukemias. The subject or individual for treatment is preferably a mammal, and more preferably a human, however it can be envisaged that any animal with a AML1/ETO-related disorder can be treated by the method of the invention.

The present invention provides kits that can be used in the above methods. In one embodiment, a kit comprises a pharmaceutical composition of the invention in one or more containers. Additionally, the can comprise instructions for use of the reagents to diagnose, predict, or monitor, an individual diagnosed with, or being tested for, a disorder associated with expression of a leukemogenic transcription factor, or it targets, as described herein.

In another embodiment, the kit is a diagnostic kit for use in testing serum samples. The kit can include a control antibody that does not react with the polypeptide of interest in addition to a specific antibody or antigen-binding fragment thereof which binds to the polypeptide (antigen) of the invention being tested for in the serum sample. Such a kit may include a substantially isolated polypeptide antigen comprising an epitope which is specifically immunoreactive with at least one anti-polypeptide antigen antibody. Further, such a kit can include a means for detecting the binding of said antibody to the antigen (for example, the antibody may be conjugated to a fluorescent compound such as fluorescein or rhodamine which can be detected by flow cytometry). In a further embodiment, the kit may include a recombinantly produced or chemically synthesized polypeptide antigen. The polypeptide antigen of the kit may also be attached to a solid support.

In an alternative embodiment, the detecting means of the above-described kit includes a solid support to which said polypeptide antigen is attached. The kit can also include a non-attached reporter-labeled anti-human antibody. Binding of the antibody to the polypeptide antigen can be detected by binding of the said reporter-labeled antibody.

In an additional embodiment, the invention includes a diagnostic kit for use in screening serum samples containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In another embodiment, the antibody may be a monoclonal antibody. The detecting means of the kit can include a second, labeled monoclonal antibody. Alternatively, or in addition, the detecting means can include a labeled, competing antigen.

In one diagnostic configuration, the test serum sample is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. Generally, the reagent is washed again to remove unbound labeled antibody, and the amount of reporter associated with the reagent is determined. The reporter can be an enzyme, for example, which is detected by incubating the solid phase in the presence of a suitable fluorometric, luminescent or calorimetric substrate, as is standard in the art.

The solid surface reagent in the above assay is prepared by known techniques for attaching protein material to solid support material. Suitable solid support materials include, for example and without limitation, polymeric beads, dip sticks, 96-well plate or filter material.

In one embodiment, a kit for diagnosing a disorder associated with leukemogenic transcription factor expression is also envisaged. The components of the kit preferably contain a reagent, or reagents, to detect the expression of ICSBP, Id1, EST similar to yeast YER036C, IL-6, or calcyclin, and combinations thereof. Such reagents can include, but without limitation to, antibodies, PCR primers, oligonucleotide probes or the like as will be recognized by one of ordinary skill in the art.

A further embodiment encompasses a kit for diagnosing a disorder associated with leukemogenic transcription factor expression, which contains a reagent to detect the expression of ICSBP, MHC class 2, B7-1, and combinations thereof.

In another embodiment, the invention provides for a method to induce the expression of IL-6 and/or calcyclin in a cell, or to inhibit the expression of ICSBP, Id1, and/or an EST similar to YER036C in a cell, by inducing the expression of TEL in said cell. Expression of TEL can be achieved by several procedures, as will be recognized by one of skill in the art, including use of transfection or infection techniques of recombinant DNA and the like.

Another embodiment provides for a method to induce the expression of ICSBP, Id1, and/or an EST similar to YER036C in a cell, or by inhibiting the expression of IL-6 and/or calcyclin, by inhibiting the expression of TEL in said cell. Expression of TEL can be inhibited in a cell by the use of antagonists, as will be recognized by one of skill in the art, and include using agents such as anti-sense oligonucleotides, ribozymes, and small molecule inhibitors and the like.

In a further embodiment, a method to increase expression of interferon gamma-induced genes, or interferon gamma-induced cytostasis, in a cell by administering to said cell ICSBP, an ICSBP agonist, an ICSBP mimic, or combinations thereof, is provided. Administration of ICSBP or agonist or mimic thereof, can be by any means known in the art, for example, by directly contacting the cell, transfection or infection techniques or the like.

Another embodiment of the invention relates to the treatment of an individual having a disorder characterized by expression of AML1/ETO or altered TEL expression, elevated or repressed expression of any one of ICSBP, MHC class 2, B7-1, Id1, calcyclin, EST similar to yeast YER036C, or IL-6, wherein an effective amount of an agent that modulates ICSBP activity is administered to the individual. Optionally, the agent can be administered in combination with a pharmaceutical carrier as is commonly used in the art. Administration of the agent may be orally, intravenously, intramuscularly, subcutaneously, topically, rectally, or by inhalation. Administration can be formulated to deliver the agent in a single dosage, multiple dosages, or to gradually infuse over time. Other methods of administration will be known to those skilled in the art. Disorders that may be treated with these agents include lymphoid, myeloid, acute and chronic leukemias. The subject or individual for treatment is preferably a mammal, and more preferably a human, however it can be envisaged that any animal with an ICSBP-related disorder can be treated by the method of the invention.

In a further embodiment, the invention relates to the treatment of an individual having a disorder characterized by repressed expression of genes normally induced by interferon gamma stimulation. Treatment of the individual comprises administering to the individual a therapeutically-effective amount of an agent selected from ICSBP, an ICSBP agonist or an ICSBP mimic, or combination thereof. In a preferred embodiment, the disorder is associated with expression of TEL, a functional fragment of TEL or a TEL fusion protein. Alternatively, the disorder is associated with the expression of AML1/ETO.

The following Examples are offered for the purpose of illustrating the present invention and are not to be construed to limit the scope of this invention. The teachings of all references cited herein are hereby incorporated herein by reference.

### EXEMPLIFICATION

Materials and Methods

Cell Lines

The murine myeloid progenitor 32Dc13 cells and the B lymphoid BaF3 cells were maintained in RPMI 1640 medium (Cellgro) supplemented with 10% fetal bovine serum (Sigma) and 50 ng/ml interleukin-3 (IL-3) (R & D Systems Inc.). For differentiation studies, granulocyte colony-stimulating factor (G-CSF) (Amgen Inc., CA) at 25 ng/ml was used to replace IL-3 in the growth medium.

Generation of Cells with Enforced TEL Expression

A human full-length TEL cDNA was subcloned into the Kpnl site of the pPC18 plasmid, which contains the human metallothionein promoter and a neomycin selection cassette. Both 32Dc13 and BaF3 cells were electroporated (200 V and 350 V respectively at 950 μF) with either pPC18 or pPC18-TEL, and stable transfectants were selected with G-418 at 800 μg/ml and 600 μg/ml respectively. Two lines of 32Dc13 and three BaF3 lines were generated that over-express TEL.

Oligonucleotide Microarray Expression Profiling.

Total RNA was isolated from 32Dc13 parental, vector alone and the two TEL over-expressing 32D lines (#1 and #12) using TRIzol® reagent according to manufacturer's specifications (Life Technologies, Gaithersburg, Md.). The RNA was amplified, labeled, and hybridized to oligonucleotide arrays as described elsewhere (Lockhart, et al. (1996) Nat Biotechnol 14, 1675-80). Each RNA sample was hybridized to a set of Affymetrix Mu6500 GeneChip®, which enabled the interrogation of 6500 murine genes and ESTs.

Northern Analysis.

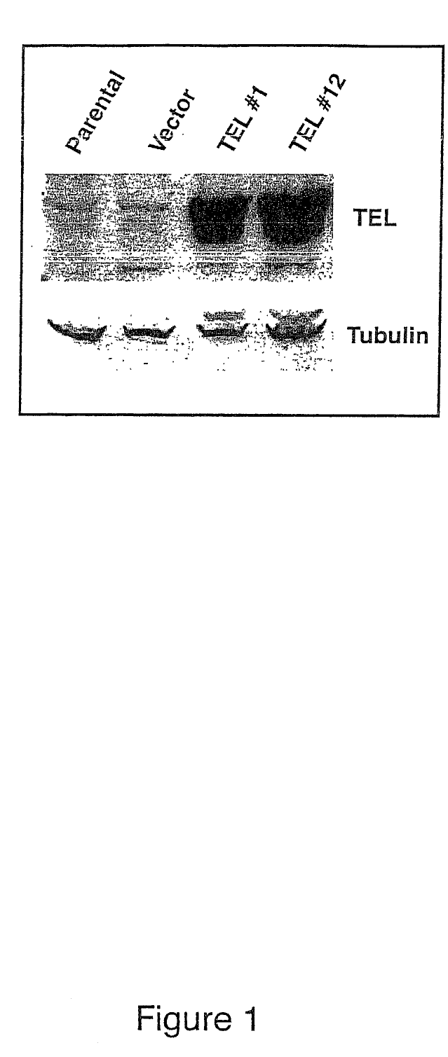

Total cytoplasmic RNA was prepared from cells plated at a density of 5×10⁵ cells/ml and harvested 24 hr thereafter. After electrophoresis through a 1.2% formaldehyde-agarose gel, RNA was transferred to Hybond™ N+ (Amersham) and subsequently probed with murine probes, as indicated in FIG. 1C, labeled by random priming.

Transient Transfection and Luciferase Assay.

Reporter plasmids that contain a 210 bp enhancer of the Idl gene (containing two putative core EBS), and plasmids that contain mutations of either EBS, denoted as m25 and m26 in the pGL2-promoter backbone, were kind gifts from R. Benezra (Memorial Sloan-Kettering Cancer Center) (Tournay and Benezra (1996) Mol Cell Biol 16, 2418-30). Approximately 1×10⁷ BaF3 cells were electroporated with the luciferase reporter plasmids (12 μg), pcDNA3 (1.2 μg) (Invitrogen, San Diego, Calif.) with or without TEL and pBluescript-KS was used to normalize the total amount of DNA to 20 μg for electroporation. Two days post-electroporation, cells were harvested and assayed for luciferase activity by using a luciferase assay kit (Promega, Madison, Wis.). The data shown represents the averages of four independent experiments, performed in triplicate.

Western Analysis.

32D and BaF3 cell lines and their derived clones were harvested for total cell lysates with a modified RIPA buffer (10 mM Tris [pH 7.6], 100 mM NaCl, 1 mM EDTA, 1% Triton X-100, 0.5% sodium deoxycholate, 0.1% SDS). Cell lysates were subsequently clarified via centrifugation (13,000 rpm for 8 min) and thereafter resolved via 7.5% SDS-PAGE and transferred to nitrocellulose membranes (Schleicher & Schuell). Western blots were performed with an anti-TEL antibody directed towards the COOH-terminal (a kind gift from Dr. O. Bernard, INSERM, Paris France), an anti-Id1 antibody (catalog #sc-427, Santa Cruz Biotechnology, Inc) and an anti-tubulin antibody (catalog #1111876, Boehringer Mannheim) followed by a 1:8000 dilution with the appropriate anti-mouse or anti-rabbit IgG antibody conjugated to horseradish peroxidase (Santa Cruz). Immune complexes were detected via enhanced chemiluminescence (NEN, DuPont) and autoradiography. Western blots were stripped by incubations at 50° C. for 30 minutes in a buffer containing 62.5 mM Tris [pH 6.8], 0.02% SDS and 10 mM beta-mercaptoethanol.

Electrophoretic Mobility Shift Assay (EMSA).

Crude nuclear extracts from different cell lines were prepared according to a Nonidet P-40 lysis procedure (Shreiber, E., et al, 1989). Mobility shifts were performed with double-stranded 31bp synthetic oligonucleotides containing either a dual tandem EBS or mutations of these EBS which were 5′-end labeled with T4 polynucleotide kinase and [γ⁻³²P]ATP. The sequences of these oligonucleotides are: wild-type EBS 5′-AGACCAGCCCGGGAAAGGAAAGGGAGGGGGT-3′ (SEQ ID NO: 2) and mutated EBS 5′-AGACCAGCCCGCTCGAGCTCAGGGAGGGGGT-3′ (SEQ ID NO: 3), the underlined bases represent the mutations within the EBS. The binding reactions were carried out in a 20 μl volume containing 12 mM HEPES (pH 7.9), 4 mM Tris-HCl (pH 7.9), 1 mM EDTA, 1 mM dithiothreitol, 12% glycerol, 2 μg poly(dI-dC), 15 μg of nuclear extract and 50,000 cpm of probe. The nuclear extract was incubated in the reaction mixture devoid of the probe for 15 min on ice, the probe was then added and the reaction was incubated at room temperature for 15 min. The reaction contents were then loaded on a 5% polyacrylamide gel (29:1, acrylamide-bisacrylamide) and run in 0.25×TBE buffer (1×TBE is 50 mM Tris, 50 mM boric acid, 1 mM EDTA) for 2.5 h at 8 V/cm. The gels were dried and autoradiographed with intensifying screens at −80° C.

Chromatin Immunoprecipitation (ChIP).

1×10⁸ 32D cells 32D/TEL were crosslinked with formaldehyde buffer (11% formaldehyde, 100 mM NaCl, 1 mM EDTA and 50 mM HEPES pH 7.5) to a final 1% concentration at room temperature. The cells were then harvested, homogenized and cleared from large debris by sucrose sedimentation. The nuclei were then re-suspended in ChIP buffer (100 mM NaCl, 60 mM KCl, 10 mM Tris-HCl pH 7.4, 0.1% NP-40 and protease inhibitors) and then sheared by sonication using a Fisher Dismembranator with microtip at setting 4 (80% of maximum) by two 1 min pulses on ice. The solutions were then cleared once by centrifugation (10 min, 14000 g, 4° C.) and then once by salmon sperm DNAIBSA blocked agarose protein A beads (Upstate Biotechnology Inc. NY). Antibodies used for immunoprecipitation (anti-TEL Pointed domain rabbit polyclonal antibody [a kind gift from Dr. Peter Marynen, University of Leuven, Leuven, Belgium], rabbit antiserum and no additive) were added to each sample and incubated on a rotator at room-temperature for 2 h. Agarose protein A-beads were then added to the samples and similarly incubated for 1 h. The agarose protein A bead conjugates were then washed once with ChIP buffer, once with 500 mM NaCl and 0.01% SDS containing CHIP buffer, once with Li-buffer (0.25M LiCl, 10 mM Tris-HCl pH 7.4, 1% NP-40 and 1% deoxycholate) and once with TE pH 8. The immunoprecipitate was then released from the beads, digested with proteinase K and then phenol/chloroform extracted in 0.6M Na Acetate (pH 8) to recover DNA. Semi-quantitative PCR was performed on each sample, using mouse Id1 210 bp enhancer primers: FWD 5′GAGAATGCTCCAGCCCAGTTTG (SEQ ID NO: 4) and REV 5′TCCGAGCAAGCTCTCCCTCC (SEQ ID NO: 5) and primers specific to regions within mouse tyrosinase promoter (a negative control for immunoprecipitated DNA), FWD 5′ GAGGCAACTATTTTAGACTGATTACTTT (SEQ ID NO: 6) and REV 5′ AGGTTAATGAGTGTCACAGACTTC (SEQ ID NO: 7). Bands were analyzed on a 1% agarose gel and visualized by ethidium bromide staining.

### Example 1

Characterization of TEL-Overexpressing 32Dc13 Cells.

The 32Dc13 cells are a well-defined murine IL-3 dependent myeloid cell line, often used to study various aspects of hematopoiesis (Valtieri, et al. (1987) J Immunol 138, 3829-35). Lines that over-express TEL (FIG. 1) showed no alteration in proliferation, apoptotic and/or differentiation rates relative to vector transfected and the parental lines. Interestingly, when lines over-expressing TEL were induced to differentiate with G-CSF, levels of both endogenous and human TEL protein were markedly decreased by day 2. These results suggest that the TEL protein is specifically targeted for destruction during the differentiation process.

### Example 2

Oligonucleotide Microarray Expression Profile.

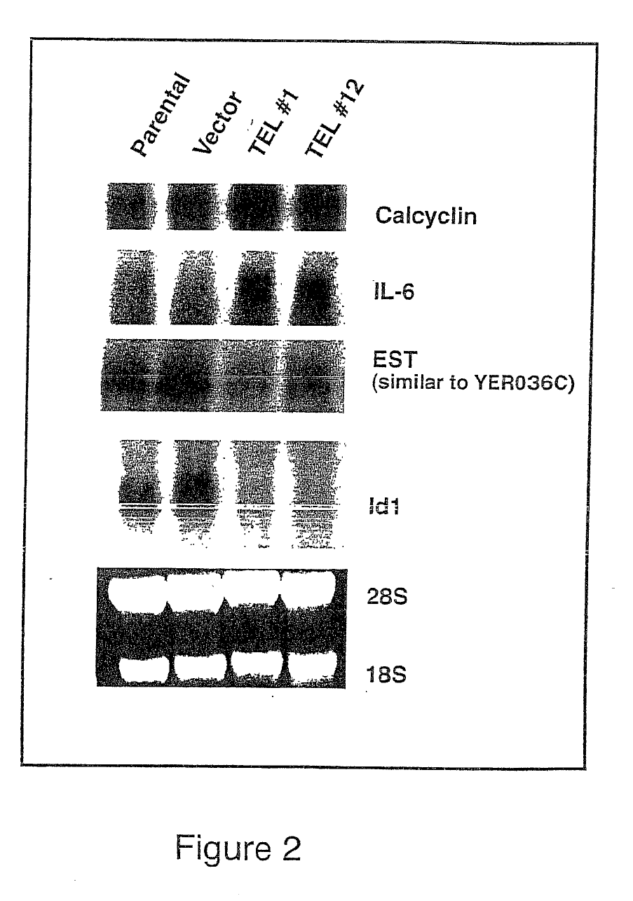

Total RNA was isolated from 32Dc13 parental, vector and two lines that over-express TEL (#1 and #12) as described. Each RNA sample was hybridized to a set of four arrays, which enabled the interrogation of 6500 murine genes and ESTs. Twenty-four candidate had a consistent three-fold change in expression between the parental and vector transfected relative to the 32D/TEL over-expressing lines. Northern blot analyses confirmed four targets that showed differential expression in the context of TEL over-expression relative to the controls. These are shown in FIG. 2.

### Example 3

TEL Represses Id1 in Both 32Dc13 and BaF3 Cells.

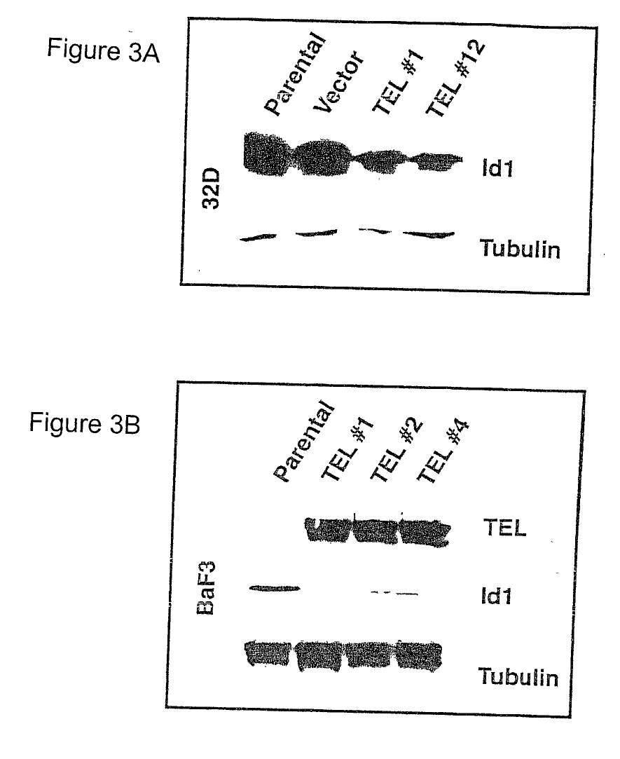

One of the genes found to be repressed in 32D/TEL cells is Id1 (FIG. 2). Id genes are members of the helix-loop-helix (HLH) family of transcription factors, which inhibit the function of basic HLH proteins by forming inactive heterodimers. Constitutive expression of Id1 in developing B cells impairs their development and contributes to lymphoma in vivo (Sun (1994) Cell 79, 893-900). It has recently been shown that Id1 and Id3 are required in neurogenesis, angiogenesis and vascularization of tumour xenografts (Lyden, et al. (1999) Nature 401, 670-7). Western blotting further confirmed diminished Id1 protein levels in TEL-over-expressing Cells (FIG. 3A). To determine whether the repressive effect of TEL on the Id1 gene is specific to 32D cells, these studies were extended to the lymphoid BaF3 cell line. As shown in FIG. 3B, BaF3 cells over-expressing TEL showed an associated reduction in Id1 protein. Thus, Id1 is a target gene of TEL in at least two hematopoietic cell lines.

### Example 4

TEL Represses the Activity of a 210-bp Enhancer Region of the Id1 Gene.

The effect of TEL expression on a luciferase reporter containing a previously described 210-bp enhancer of the Id1 gene coupled to a minimal promoter (Toumay and Benezra (1996) Mol Cell Biol 16, 2418-30) was investigated. Within the 210-bp enhancer region there is a dual tandem repeat of 5′-GGAA-3′ (SEQ ID NO: 8) representing a putative EBS. TEL reproducibly repressed the activity of the 210 bp enhancer region by three-fold following co-transfection into BaF3 cells (FIG. 4). Mutation of either putative EBS revealed a dramatic loss of the enhancer's basal activity. This suggested that there is a previously unrecognized endogenous transcription factor, potentially of the Ets family, which is required for expression of the Id1 gene. Additionally, a TEL mutant devoid of the DNA-Binding Domain (TELΔDBD) is incapable of repressing the 210 bp enhancer (FIG. 4), indicating that the repressive effect of TEL requires DNA binding.

### Example 5

TEL Induces the Formation of an Ets-Specific Complex at the EBS of the 210 bp Id1 Enhancer.

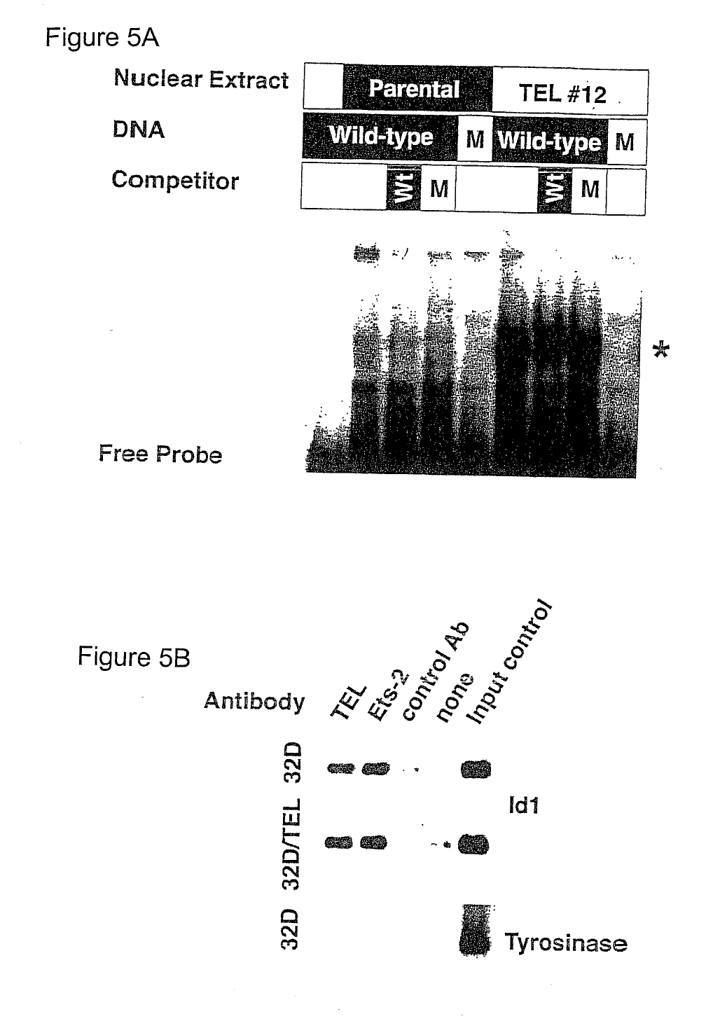

In order to determine whether specific complexes are formed between oligonucleotides containing the Id1 EBS and nuclear extracts from 32D and 32D/TEL lines, EMSA was performed. The results in FIG. 5A demonstrate the formation of a specific complex that is dependent on an intact EBS, and that is preferentially formed in the presence of TEL over-expression. A complex is seen from nuclear extracts of the parental 32D cells but this requires prolonged exposure to autoradiographic film. Unlabelled wild type, but not EBS mutant competitor diminished the intensity of the band thereby demonstrating specificity of the complex.

### Example 6

Chromatin Immunoprecipitation Identifies Id1 as a TEL Target.

In order to determine whether TEL binds the endogenous Id1 enhancer in vivo, chromatin immunoprecipitaion was performed. As shown in FIG. 5B, PCR specific for the Id1 enhancer region containing the EBS, revealed that this region was specifically amplified in eluates from anti-TEL antiserum in 32D and 32D/TEL, whereas the Id1 enhancer was not immunoprecipitated by control antiserum. In addition, anti-TEL antisera did not immunoprecipitate genomic DNA from the tyrosinase gene, used as a negative control. Taken together, these observations support the notion that TEL induces a complex that binds to the EBS within the Id1 enhancer, resulting in Id1 repression.

### Example 7

TEL Represses ICSBP Expression.

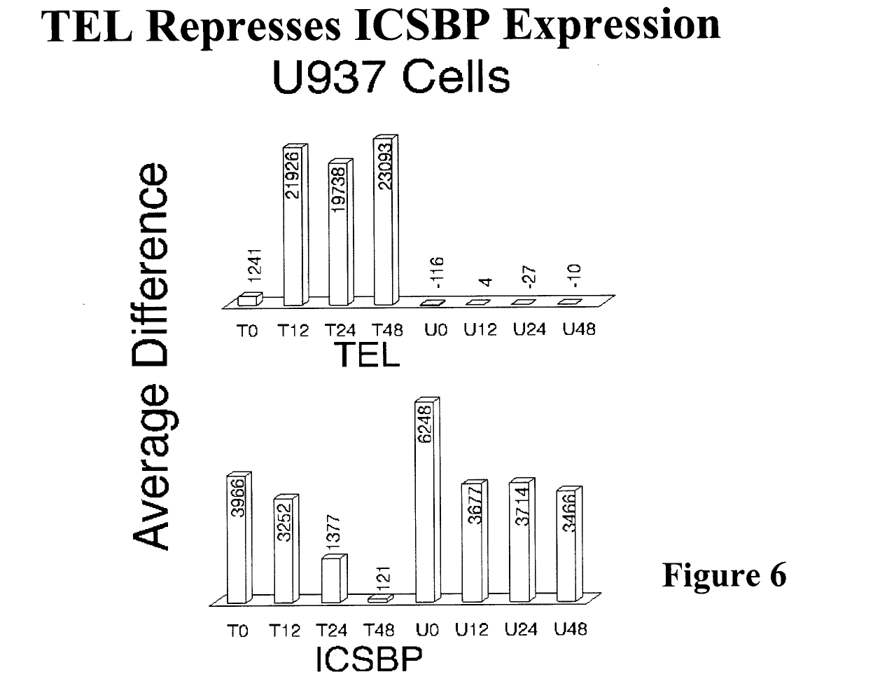

U937 cells induced to express TEL, repress ICSBP expression following interferon gamma stimulation, as compared to control treated U937 cells (FIG. 6).

### Example 8

TEL Represses Interferon Gamma-Induced ICSBP Induction.

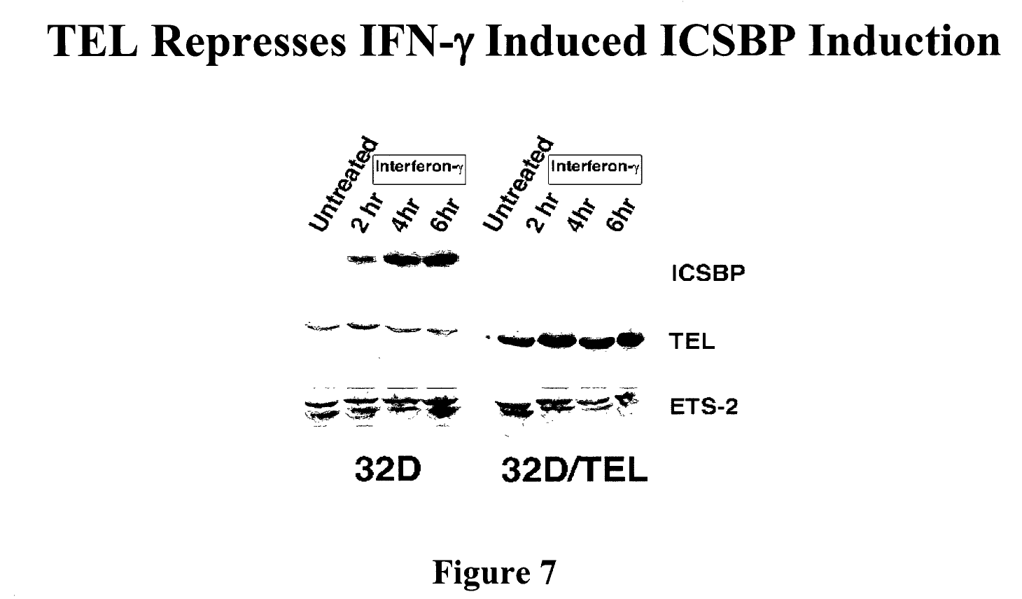

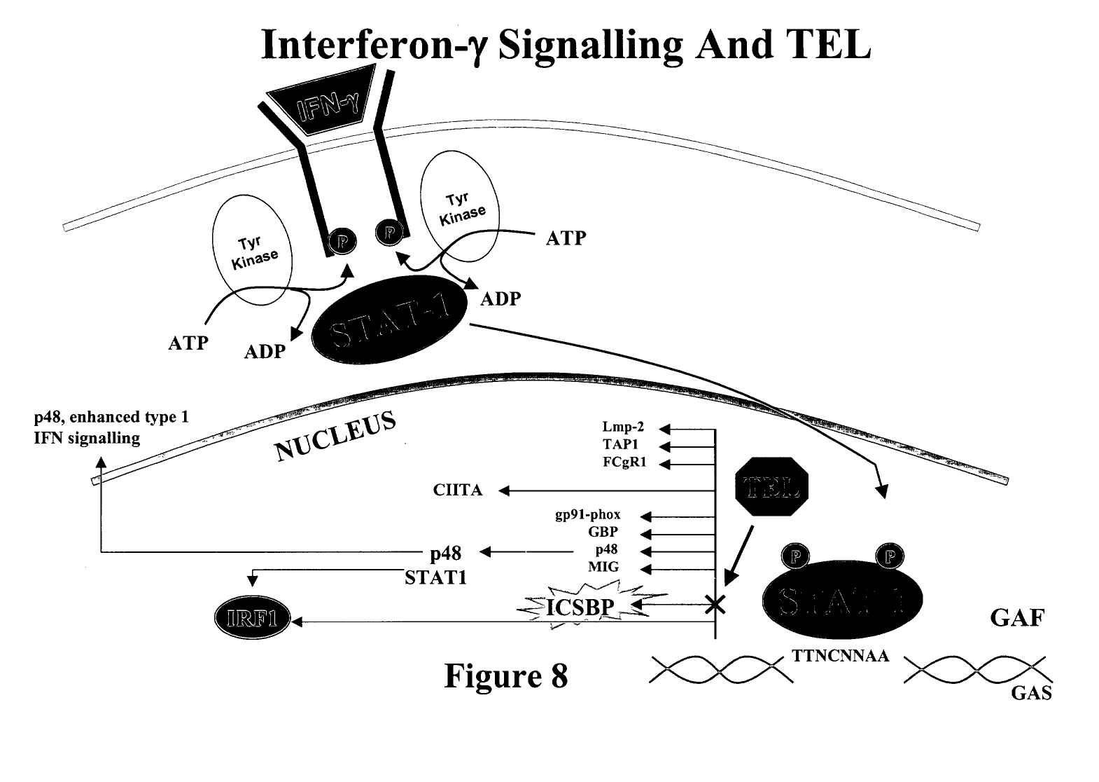

Parental 32D cells and TEL-expressing 32D cells were treated with interferon gamma and the expression of ICSBP determined. 32D cells strongly induce ICSBP expression, whereas 32D/TEL cells do not induce ICSBP expression. Over-expression of TEL was confirmed in 32D/TEL cells (FIG. 7).

### Example 9

STAT-1 Phosphorylation is Unaffected by TEL.

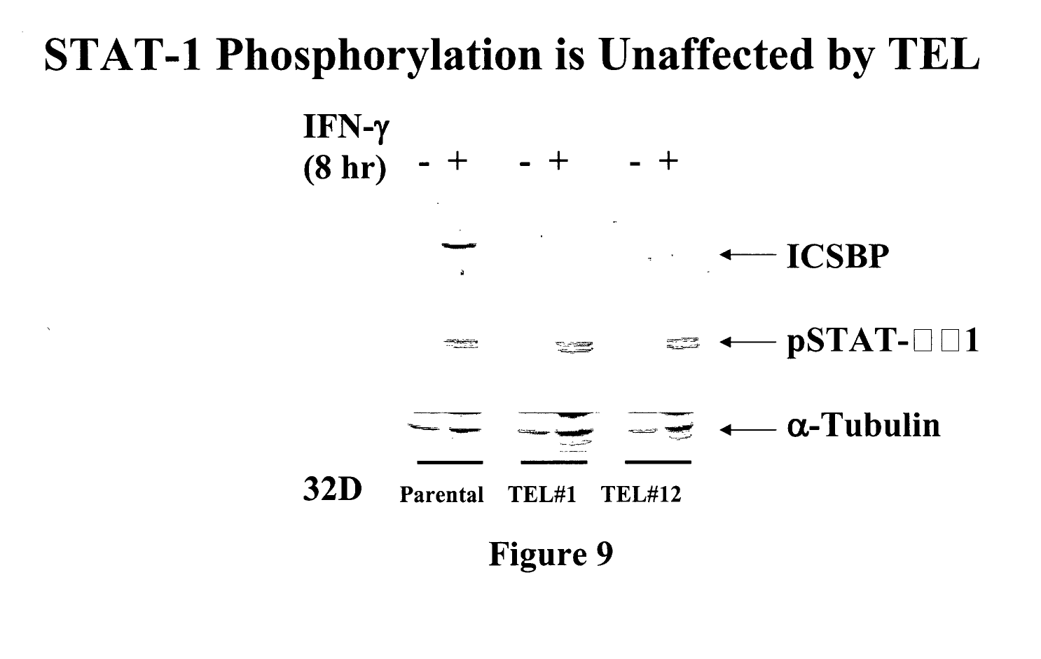

Parental and TEL-overexpressing 32D cell lines #1 and #12 were tested in the presence and absence of interferon gamma for STAT-1 phosphorylation. No difference was detected in the phosphorylation status of STAT-1, whether TEL was expressed or not. Tubulin was used as a loading control (FIG. 9).

### Example 10

Identification ofEBS in the 5′ Flanking Region of the Mouse ICSBP Gene.

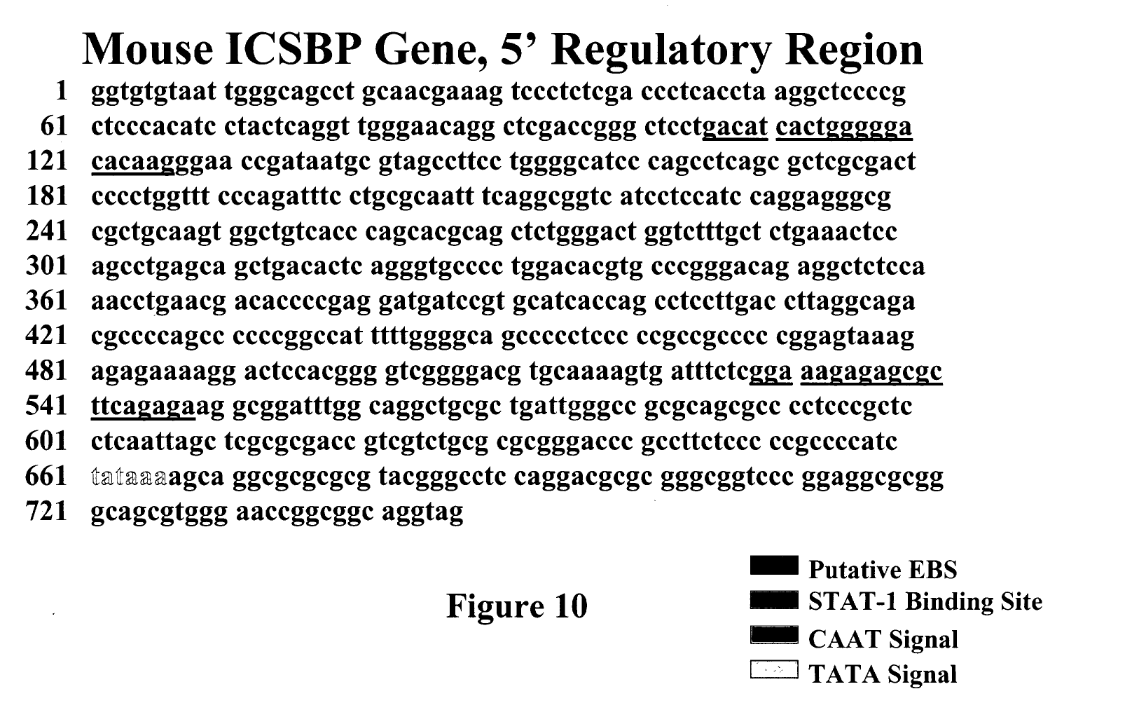

Sequence analysis revealed the sequence of the 5′ flanking region of the mouse ICSBP gene to contain two putative EBS (Ets-binding sequences), consensus STAT-1 binding site, CAAT signal and TATA signal sequences; SEQ ID NO: 1 (FIG. 10).

### Example 11

TEL Represses the Activity of the 5′ ICSBP Flanking Region.

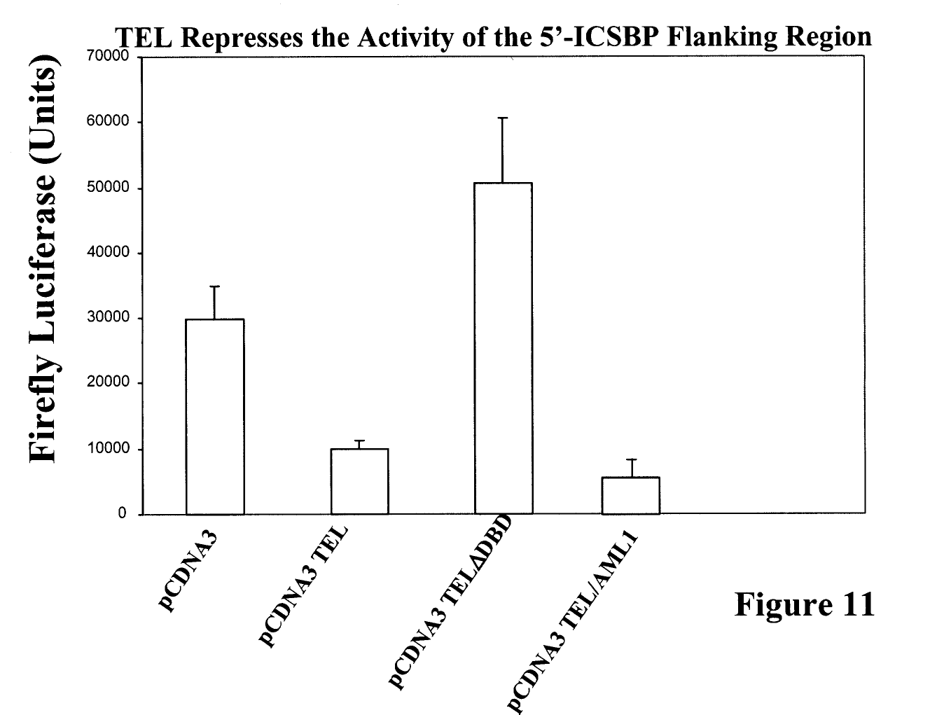

Using a Luciferase assay, cells repress the activity of the 5′ ICSBP flanking region comprising SEQ NO: 1, when TEL is present, as compared to control pcDNA3. This repression by TEL is dependent on the DNA-binding domain (DBD) as transfection with a TEL construct without the DBD fails to repress activity of the 5′ ICSBP flanking region. TEL/AML1 also represses the activity of the 5′ ICSBP flanking region when compared to control pcDNA3 (FIG. 11).

### Example 12

TEL Repression of ICSBP is Due to Specific Deacetylation of Histone H3.

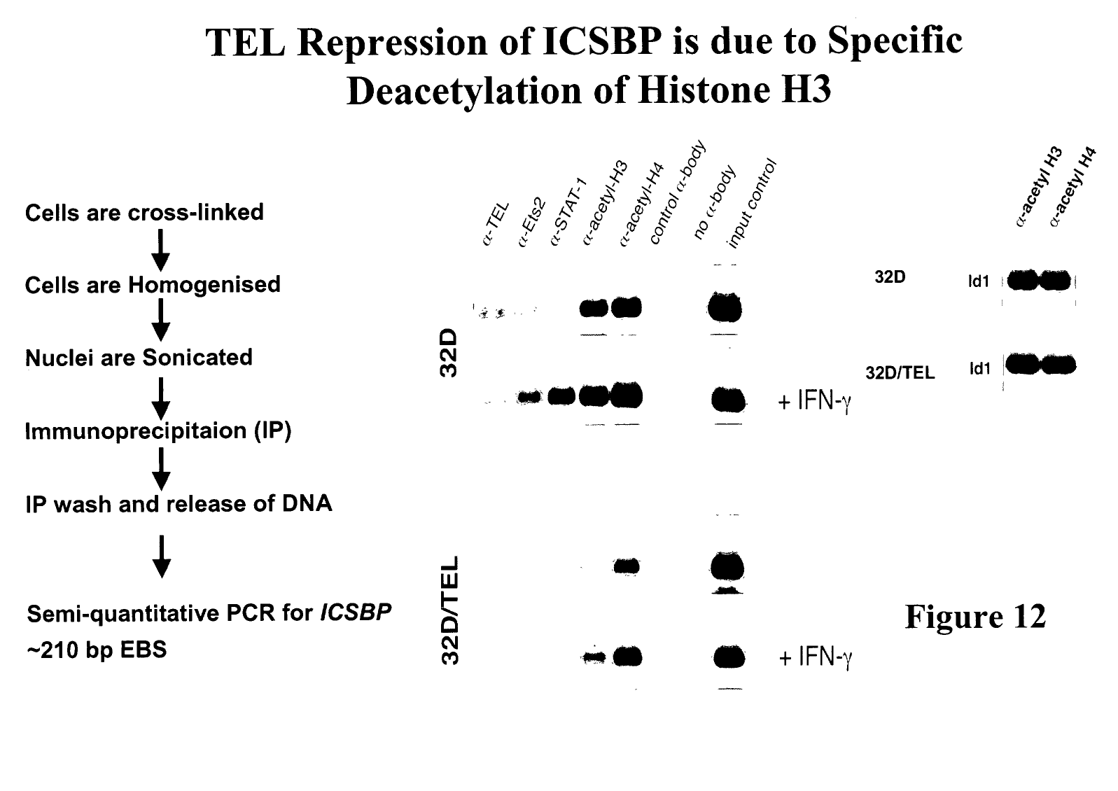

Parental 32D cells or 32D/TEL cells in the presence or absence of interferon-gamma were subjected to cross-linking, homogenization, and the nuclei sonicated. Immunoprecipitations were performed with antibodies to TEL, Ets2, STAT-1, acetylated Histone H3, acetylated Histone H4, a control antibody, or without antibody. Immunoprecipitates were washed, the DNA eluted, and subjected to semi-quantitative polymerase chain reaction (PCR) for a region of the ICSBP EBS-containing sequence. Acetylated Histone H3 was associated with the EBS-containing region of ICSBP in parental 32D cells, but not in TEL expressing cells. TEL expression also inhibits the interferon gamma-induced STAT-1 association with the EBS-containing region of ICSBP (FIG. 12)

### Example 13

TEL Suppresses the Cytostatic Effect of Interferon Gamma in 32D Myeloid Cells.

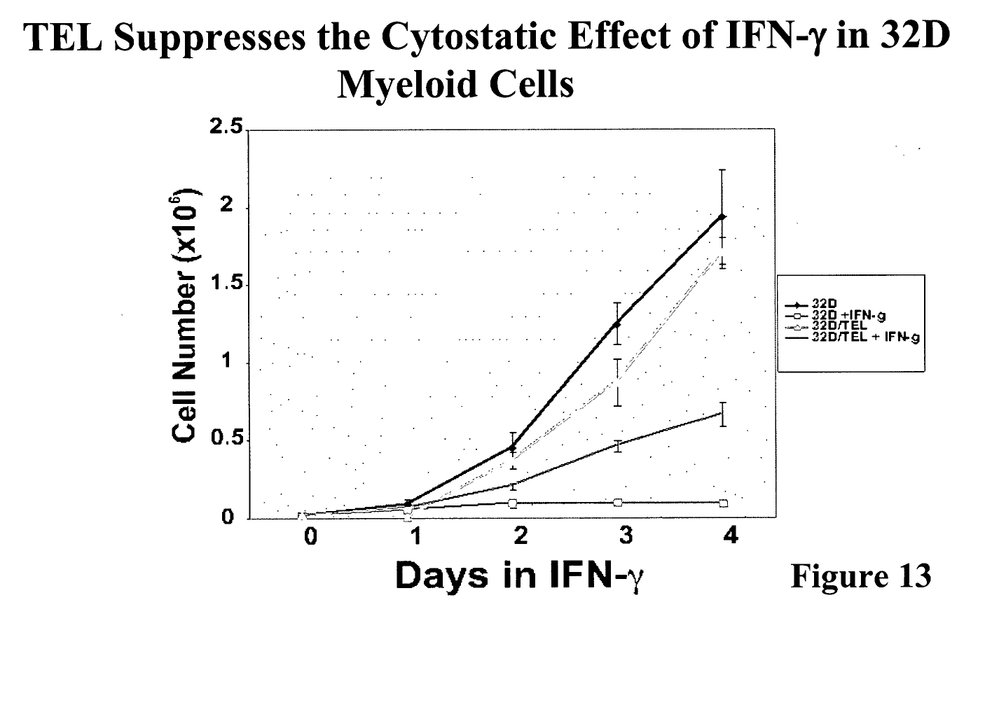

After four days in culture, TEL-expressing cells can be seen to suppress the cytostatic effect of interferon gamma, as compared to parental 32D cells (FIG. 13).

### Example 14

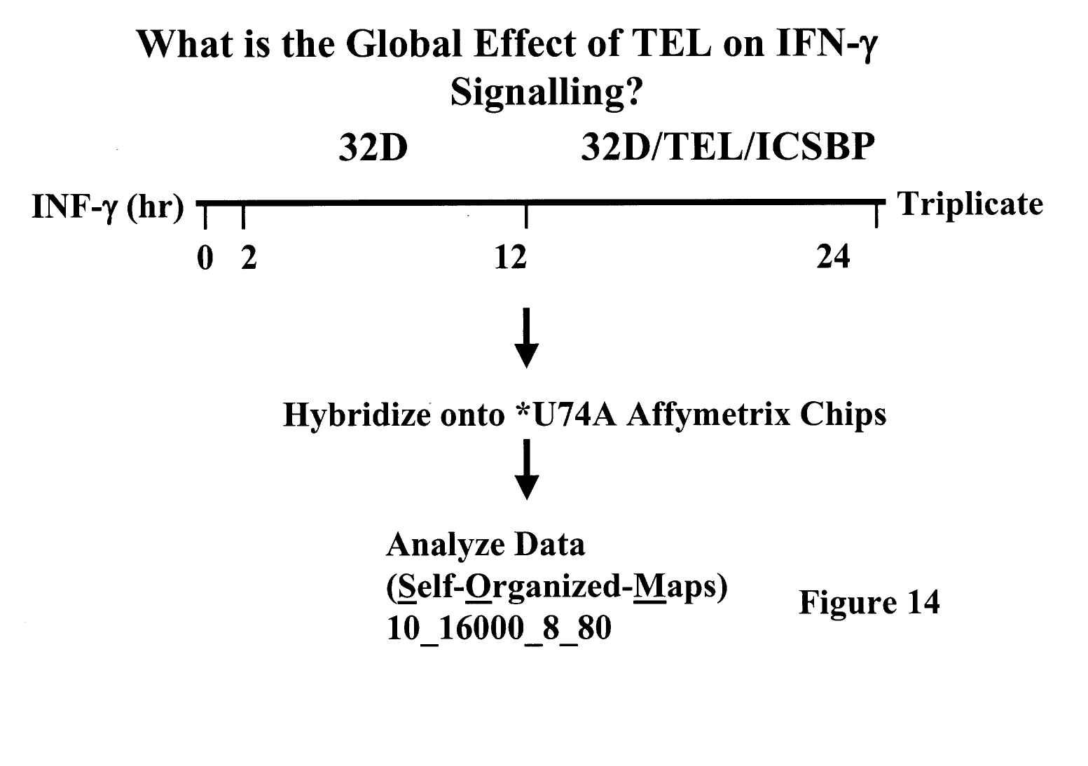

Murine genome U74A Affymetrix gene chips were used to analyze the global effect of TEL on interferon gamma signaling (FIG. 14).

### Example 15

TEL Represses Interferon Gamma-Dependent ICSBP Induction.

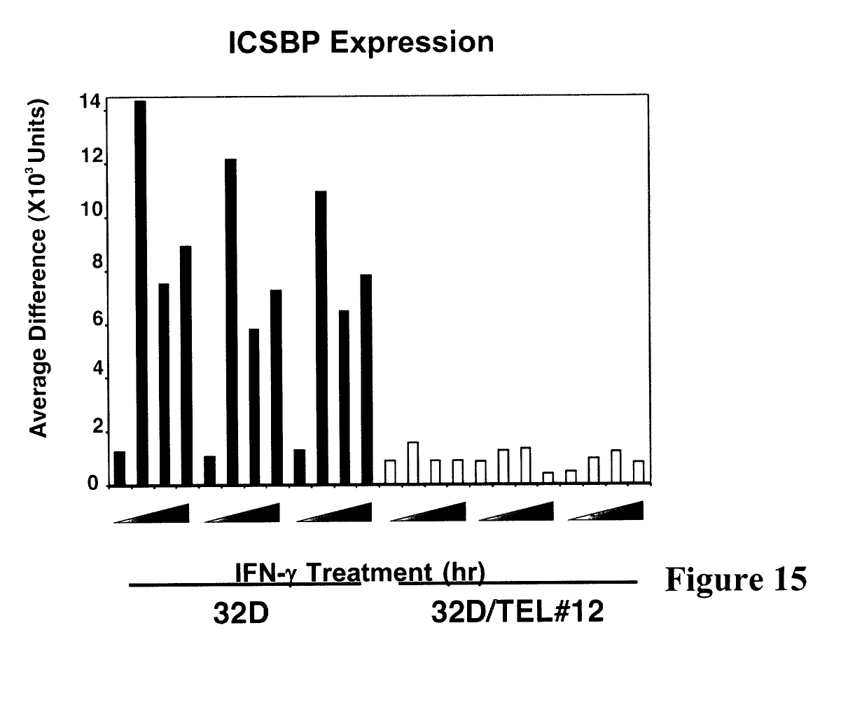

In 32D cells, ICSBP is rapidly induced upon interferon gamma-dependent treatment. TEL suppresses the interferon gamma-dependent ICSBP induction (FIG. 15).

### Example 16

TEL Significantly Affects Interferon Gamma-Induced Genes.

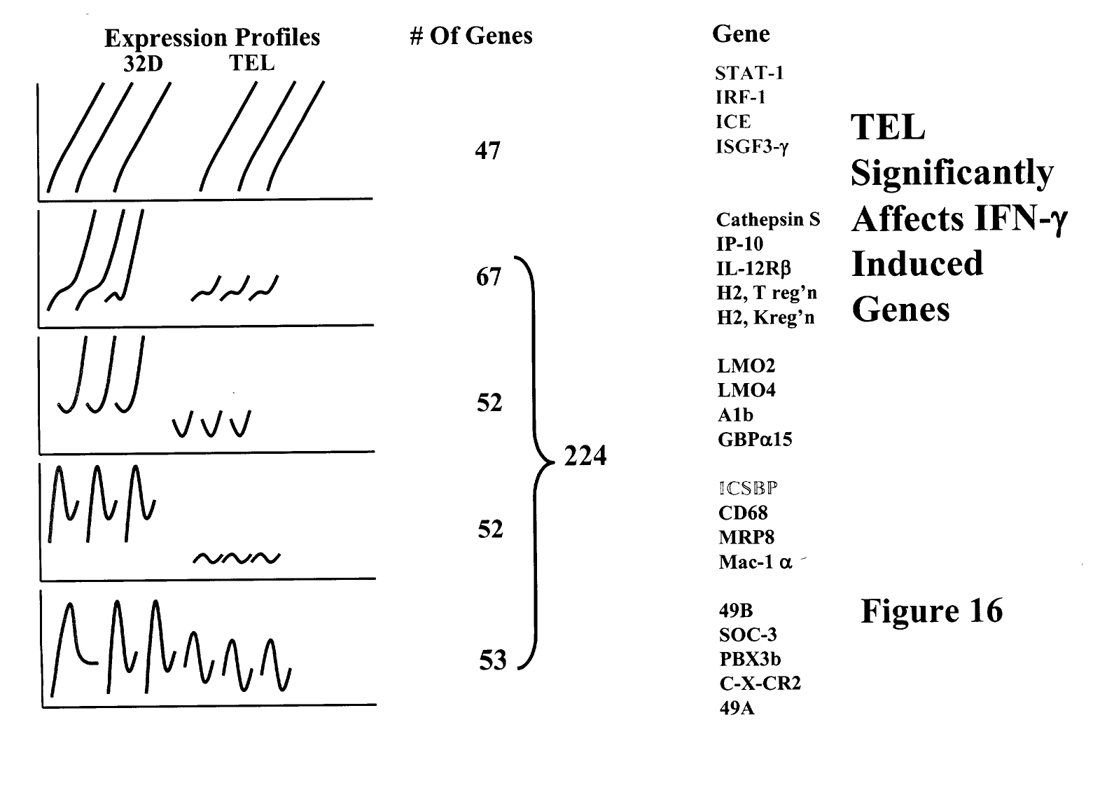

The expression profile of several genes were analyzed in 32D and 32D/TEL cells and compared. Two hundred and twenty four genes normally induced by interferon gamma treatment were suppressed in TEL-expressing cells, including ICSBP (FIG. 16).

### Example 17

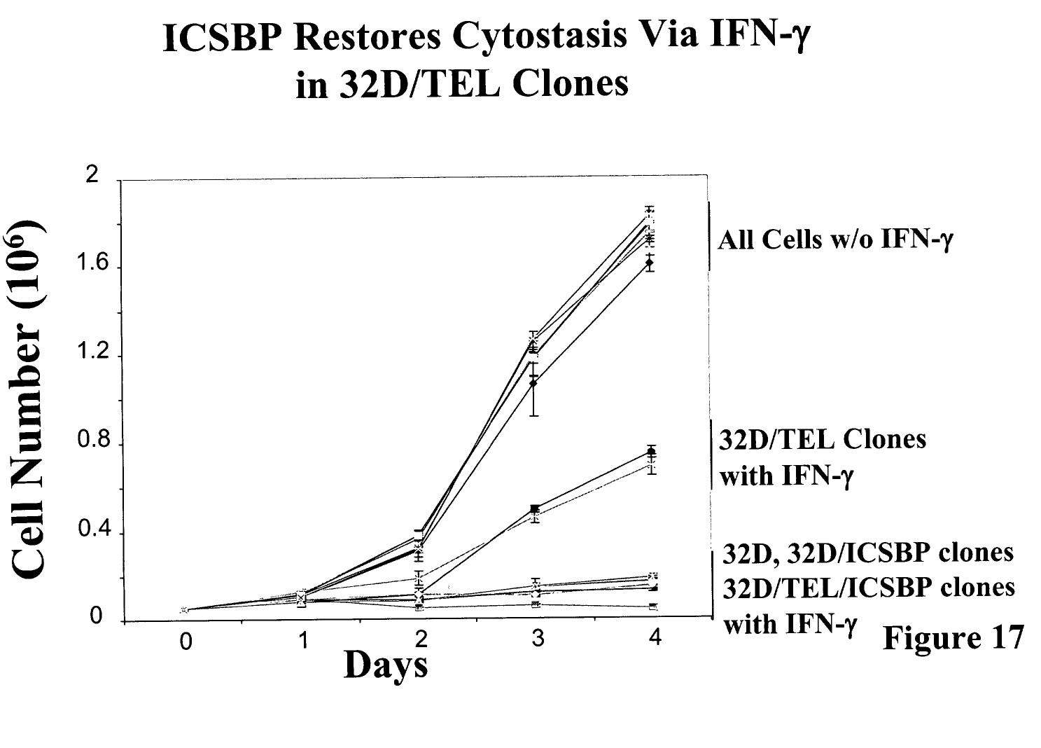

Expression of ICSBP in 32D/TEL cells restores interferon gamma-induced cytostasis (FIG. 17).

### Example 18

ICSBP expression in 32D/TEL cells rescues 100% of TEL-repressed interferon gamma-induced genes.

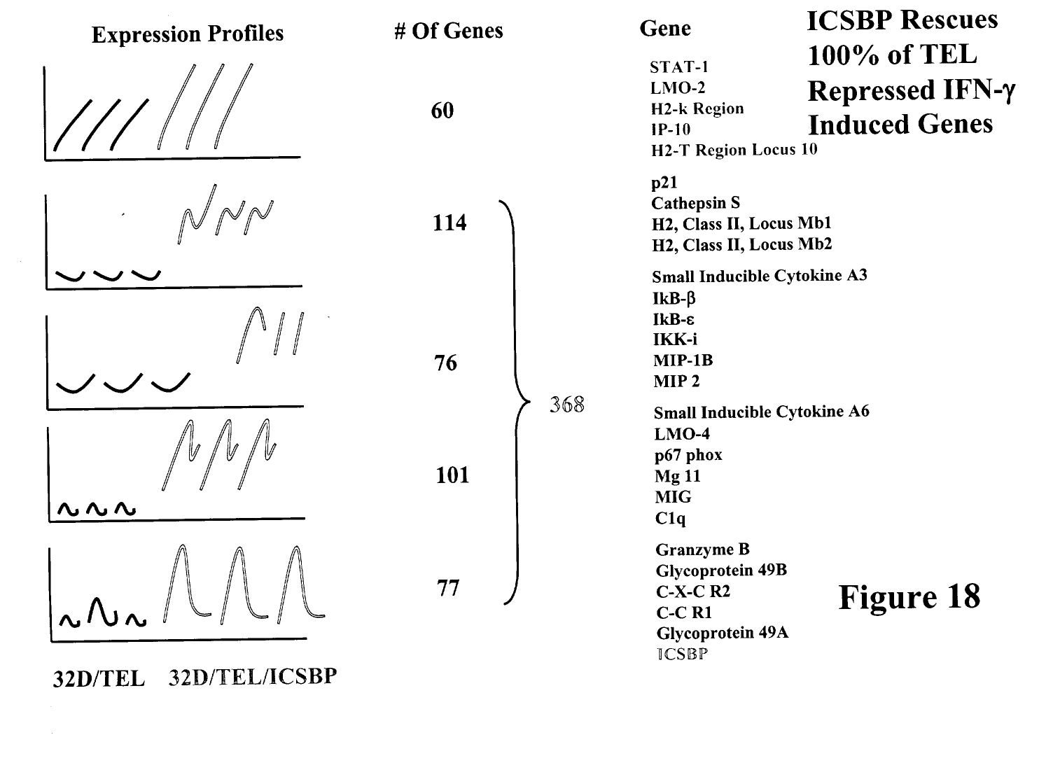

Three hundred and sixty-eight genes normally repressed in TEL-expressing cells were induced in response to interferon gamma in 32D/TEL/ICSBP cells, including ICSBP (FIG. 18).

While this invention has been particularly shown and described with references to preferred embodiments thereof, it will be understood by those skilled in the art that various changes in form and details may be made therein without departing from the scope of the invention encompassed by the appended claims.

The relevant teachings of all the references, patents and patent applications cited herein are incorporated herein by reference in their entirety.

What is claimed is:

1. A method for diagnosing a disorder associated with altered leukemogenic transcription factor expression, comprising determining the expression level of one or more target genes of said leukemogenic transcription factor.

2. The method according to claim 1, wherein the leukemogenic transcription factor is TEL, a functional fragment of TEL, or a TEL fusion protein, and the one or more target genes is selected from the group consisting of ICSBP, Id1, IL-6, calcyclin and an EST similar to yeast YER036C.

3. The method according to claim 1, wherein the leukemogenic transcription factor is AML1/ETO, and the one or more target genes is selected from the group consisting of: ICSBP, MHC class 2, and B7-1.

4. A method of screening for an agent that is an agonist, mimic, or antagonist of TEL, comprising the steps of:
(a) culturing cells that express TEL in a suitable medium;
(b) introducing an agent to be tested to TEL-expressing cells cultured in said medium;
(c) assaying TEL-expressing cells for altered expression of ICSBP, Id1, IL-6, calcyclin or EST similar to yeast YER036C; and
(d) culturing control cells, which do not express TEL, in a suitable medium, introducing same said agent to be tested, and assaying said control cells for expression of ICSBP, Id1, IL-6, calcyclin or EST similar to yeast YER036C;
wherein repressed expression of ICSBP, Id1, EST similar to yeast YER036C, or increased expression of IL-6 or calcyclin in cells that express TEL and treated with the agent, relative to control cells treated with the agent, indicates that the agent is an agonist or mimic of TEL; whereas increased expression of ICSBP, Id1, EST similar to yeast YER036C, or repressed expression of IL-6 or calcyclin in cells that express TEL and treated with the agent, relative to control cells treated with the agent, indicates that the agent is an antagonist of TEL.

5. A method for determining the effectiveness of an agent that modulates TEL activity for treatment of a disorder characterized by altered TEL expression, comprising determining the differentiation of test cells that have altered TEL expression, cultured in a suitable culture medium in the presence and absence of the agent to be tested, wherein the test cells are obtained from the subject, and increased differentiation of test cells in the presence of the agent, relative to test cells in the absence of the agent, is predictive of the efficacy of the agent for the treatment of the disorder.

6. A method of screening for an agent that is an agonist, mimic, or antagonist of AML1/ETO, comprising the steps of:
(a) culturing cells that express AML1/ETO in a suitable medium;
(b) introducing an agent to be tested to AML1/ETO-expressing cells cultured in said medium;
(c) assaying AML1/ETO-expressing cells for altered expression of ICSBP, MHC class 2, or B7-1; and
(d) culturing control cells, which do not express AML1/ETO, in a suitable medium, introducing same said agent to be tested, and assaying said control cells for expression of ICSBP, MHC class 2, or B7-1;
wherein repressed expression of ICSBP, MHC class 2, or B7-1 in cells that express AML1/ETO and treated with the agent, relative to control cells treated with the agent, indicates that the agent is an agonist or mimic of AML1/ETO; whereas increased expression of ICSBP, MHC class 2, or B7-1 in cells that express AML1/ETO and treated with the agent, relative to control cells treated with the agent, indicates that the agent is an antagonist of AML1/ETO.

7. A method for determining the effectiveness of an agent that modulates AML1/ETO activity for treatment of a disorder characterized by altered AML1/ETO expression, comprising determining the differentiation of test cells that have altered AML1/ETO expression, cultured in a suitable culture medium in the presence and absence of the agent to be tested, wherein the test cells are obtained from the subject, and increased differentiation of test cells in the presence of the agent, relative to test cells in the absence of the agent, is predictive of the efficacy of the agent for the treatment of the disorder.

8. A method of treatment of an individual having a disorder characterized by one or more parameters selected from the group consisting of:
(a) elevated or repressed TEL gene expression;
(b) expression of TEL protein, or fragment thereof, fused to another protein as a consequence of chromosomal translocation;
(c) elevated or repressed ICSBP gene expression;
(d) elevated or repressed Id1 gene expression;
(e) elevated or repressed calcyclin gene expression;
(f) elevated or repressed EST similar to yeast YER036C gene expression, and
(g) elevated or repressed IL-6 gene expression;
said method comprising administering to the individual an effective amount of an agent that modulates TEL activity.

9. A method of treatment of an individual having a disorder characterized by one or more parameters selected from the group consisting of:
(a) expression of AML1/ETO;
(b) elevated or repressed ICSBP gene expression;
(c) elevated or repressed MHC class 2 gene expression; and
(d) elevated or repressed B7-1 gene expression;
said method comprising administering to the individual an effective amount of an agent that modulates AML1/ETO activity.

10. A method for diagnosing a disorder associated with altered leukemogenic transcription factor expression, comprising determining the expression level of one or more target genes of said leukemogenic transcription factor, wherein the disorder is selected from the group consisting of lymphoid leukemia, myeloid leukemia, acute leukemia and chronic leukemia.

11. A method for diagnosing a disorder associated with altered leukemogenic transcription factor expression, wherein altered leukemogenic transcription factor expression results in dysregulation of the interferon gamma transcriptional response.

12. A kit for diagnosis of a disorder associated with altered leukemogenic transcription factor expression comprising one or more reagents to detect the expression of ICSBP, Id1, EST similar to yeast YER036C, IL-6, calcyclin, MHC class 2, B7-1, and combinations thereof.

13. A method for inducing expression in a cell of a gene selected from the group consisting of IL-6 and calcyclin, comprising inducing the expression of TEL in said cell.

14. A method for inhibiting expression in a cell of a gene selected from the group consisting of ICSBP, Id1, an EST similar to yeast YER036C, comprising inducing the expression of TEL in said cell.

15. A method for inducing expression in a cell of a gene selected from the group consisting of ICSBP, Id1, an EST similar to yeast YER036C, comprising inhibiting the expression of TEL in said cell.

16. A method for inhibiting expression in a cell of a gene selected from the group consisting of IL-6 and calcyclin, comprising inhibiting the expression of TEL in said cell.

17. A method for increasing expression of interferon gamma-induced genes in a cell comprising administering to said cell an agent selected from the group consisting of ICSBP, an ICSBP mimic, an ICSBP agonist, and combinations thereof.

18. A method for inducing interferon gamma-induced cytostasis in a cell comprising administering to said cell an agent selected from the group consisting of ICSBP, an ICSBP mimic, an ICSBP agonist, and combinations thereof.

19. A method of treatment of an individual having a disorder characterized by one or more parameters selected from the group consisting of:
(a) elevated or repressed TEL gene expression;
(b) expression of TEL protein, or fragment thereof, fused to another protein as a consequence of chromosomal translocation;
(c) elevated or repressed ICSBP gene expression;
(d) elevated or repressed Id1 gene expression;
(e) elevated or repressed calcyclin gene expression;
(f) elevated or repressed EST similar to yeast YER036C gene expression;
(g) expression of AML1/ETO;
(h) elevated or repressed MHC class 2 gene expression;
(i) elevated or repressed B7-1 gene expression; and
(j) elevated or repressed IL-6 gene expression;
wherein said method comprising administering to the individual an effective amount of an agent that modulates ICSBP activity.


SEQUENCE LISTING (8 sequences)

SEQ ID NO: 1 - DNA, 746 bp, Unknown
  5′ Flanking region of the mouse ICSBP gene
ggtgtgtaattgggcagcctgcaacgaaagtccctctcgaccctcacctaaggctccccgctcccacatcctactcaggttgggaacaggctcgaccgggctcctgacatcactgggggacacaagggaaccgataatgcgtagccttcctggggcatcccagcctcagcgctcgcgactcccctggtttcccagatttcctgcgcaatttcaggcggtcatcctccatccaggagggcgcgctgcaagtggctgtcacccagcacgcagctctgggactggtctttgctctgaaactccagcctgagcagctgacactcagggtgcccctggacacgtgcccgggacagaggctctccaaacctgaacgacaccccgaggatgatccgtgcatcaccagcctccttgaccttaggcagacgccccagccccccggccatttttggggcagccccctcccccgccgcccccggagtaaagagagaaaaggactccacggggtcggggacgtgcaaaagtgatttctcggaaagagagcgcttcagagaaggcggatttggcaggctgcgctgattgggccgcgcagcgcccctcccgctcctcaattagctcgcgcgaccgtcgtctgcgcgcgggacccgccttctcccccgccccatctataaaagcaggcgcgcgcgtacgggcctccaggacgcgcgggcggtcccggaggcgcgggcagcgtgggaaccggcggcaggtag

SEQ ID NO: 2 - DNA, 31 bp, Artificial Sequence
  Oligonucleotide
agaccagcccgggaaaggaaagggagggggt

SEQ ID NO: 3 - DNA, 31 bp, Artificial Sequence
  Oligonucleotide
agaccagcccgctcgagctcagggagggggt

SEQ ID NO: 4 - DNA, 22 bp, Artificial Sequence
  Primer
gagaatgctccagcccagtttg

SEQ ID NO: 5 - DNA, 20 bp, Artificial Sequence
  Primer
tccgagcaagctctccctcc

SEQ ID NO: 6 - DNA, 28 bp, Artificial Sequence
  Primer
gaggcaactattttagactgattacttt

SEQ ID NO: 7 - DNA, 24 bp, Artificial Sequence
  Primer
aggttaatgagtgtcacagacttc

SEQ ID NO: 8 - DNA, 4 bp, Unknown
  Repeat sequence
ggaa